SEQUENCE LISTING (28 sequences)

SEQ ID NO: 1 - protein, 30 aa, Echinus esculentus
GWWRRTVDKVRNAGRKVAGFASKACGALGH

SEQ ID NO: 2 - protein, 13 aa, Echinus esculentus
DIGKYCGYAHALN

SEQ ID NO: 3 - protein, 12 aa, Artificial Sequence
  Peptide sequence
AWWRRTVAKVRK

SEQ ID NO: 4 - protein, 12 aa, Artificial Sequence
  Peptide sequence
GAWRRTVAKVRK

SEQ ID NO: 5 - protein, 12 aa, Artificial Sequence
  Peptide sequence
GWARRTVAKVRK

SEQ ID NO: 6 - protein, 12 aa, Artificial Sequence
  Peptide sequence
GWWARTVAKVRK

SEQ ID NO: 7 - protein, 12 aa, Artificial Sequence
  Peptide sequence
GWWRATVAKVRK

SEQ ID NO: 8 - protein, 12 aa, Artificial Sequence
  Peptide sequence
GWWRRAVAKVRK

SEQ ID NO: 9 - protein, 12 aa, Artificial Sequence
  Peptide sequence
GWWRRTAAKVRK

SEQ ID NO: 10 - protein, 12 aa, Artificial Sequence
  Peptide sequence
GWWRRTVAKVRK

SEQ ID NO: 11 - protein, 12 aa, Artificial Sequence
  Peptide sequence
GWWRRTVAAVRK

SEQ ID NO: 12 - protein, 12 aa, Artificial Sequence
  Peptide sequence
GWWRRTVAKARK

SEQ ID NO: 13 - protein, 12 aa, Artificial Sequence
  Peptide sequence
GWWRRTVAKVAK

SEQ ID NO: 14 - protein, 12 aa, Artificial Sequence
  Peptide sequence
GWWRRTVAKVRA

SEQ ID NO: 15 - protein, 16 aa, Artificial Sequence
  Peptide sequence
GWWRRTVDKVRNAGRK

SEQ ID NO: 16 - protein, 16 aa, Artificial Sequence
  Peptide sequence
GWWRRTVAKVRNAGRK

SEQ ID NO: 17 - protein, 15 aa, Artificial Sequence
  Peptide sequence
WWRRTVAKVRNAGRK

SEQ ID NO: 18 - protein, 12 aa, Artificial Sequence
  Peptide sequence
GWWRRTVAKVRN

SEQ ID NO: 19 - protein, 9 aa, Artificial Sequence
  Peptide sequence
GWWRRTVRK

SEQ ID NO: 20 - protein, 12 aa, Artificial Sequence
  Peptide sequence
GWWRRWVAKVRK

SEQ ID NO: 21 - protein, 12 aa, Artificial Sequence
  Peptide sequence
GWWRRKVAKVRK

SEQ ID NO: 22 - protein, 12 aa, Artificial Sequence
  Peptide sequence
GWWRRWVKKVRK

SEQ ID NO: 23 - protein, 12 aa, Artificial Sequence
  Peptide sequence
KWWRRWVKKVRK

SEQ ID NO: 24 - protein, 12 aa, Artificial Sequence
  Peptide sequence
GWARRWVAKVRK

SEQ ID NO: 25 - protein, 12 aa, Artificial Sequence
  Peptide sequence
RWWRRWVRRVRR

SEQ ID NO: 26 - protein, 12 aa, Artificial Sequence
  Peptide sequence
  misc_feature (1)..(1): an amino acid that has a hydrophobicity that is less than or equal to the hydrophobicity of glycine and is not an anionic amino acid
  misc_feature (2)..(3): an amino acid with a hydrophobic R group, said R group having at least 4 non-hydrogen atoms
  misc_feature (4)..(5): a cationic amino acid
  misc_feature (6)..(6): an amino acid that is not an anionic amino acid
  misc_feature (7)..(7): an amino acid with a hydrophobic R group, said R group having at least 3 non-hydrogen atoms
  misc_feature (8)..(8): an amino acid that is not an anionic amino acid
  misc_feature (9)..(9): a cationic amino acid
  misc_feature (10)..(10): an amino acid that is not an anionic amino acid
  misc_feature (11)..(12): a cationic amino acid
XXXXXXXXXXXX

SEQ ID NO: 27 - protein, 12 aa, Artificial Sequence
  Peptide sequence
  misc_feature (1)..(1): an amino acid that has a hydrophobicity that is less than or equal to the hydrophobicity of glycine and is not an anionic amino acid
  misc_feature (2)..(3): an amino acid with a hydrophobic R group, said R group having at least 4 non-hydrogen atoms
  misc_feature (4)..(5): a cationic amino acid
  misc_feature (6)..(6): an uncharged amino acid
  misc_feature (7)..(7): is an amino acid with a hydrophobic R group, said R group having at least 3 non-hydrogen atoms
  misc_feature (8)..(8): an amino acid that is not an anionic amino acid
  misc_feature (9)..(9): a cationic amino acid
  misc_feature (10)..(10): an amino acid that is not an anionic amino acid
  misc_feature (11)..(12): a cationic amino acid
XXXXXXXXXXXX

SEQ ID NO: 28 - protein, 12 aa, Artificial Sequence
  Peptide sequence
  misc_feature (1)..(1): an amino acid that has a hydrophobicity that is less than or equal to the hydrophobicity of glycine and is not an anionic amino acid
  misc_feature (2)..(3): an amino acid with a hydrophobic R group, said R group having at least 4 non-hydrogen atoms
  misc_feature (4)..(5): a cationic amino acid
  misc_feature (6)..(6): not an anionic amino acid
  misc_feature (7)..(7): an amino acid with a hydrophobic R group, said R group having at least 3 non-hydrogen atoms
  misc_feature (8)..(8): an amino acid that is not an anionic amino acid
  misc_feature (9)..(9): a cationic amino acid
  misc_feature (10)..(10): an amino acid with a hydrophobic R group, said R group having at least 3 non-hydrogen atoms
  misc_feature (11)..(12): a cationic amino acid
XXXXXXXXXXXX

(19) United States
(12) Patent Application Publication
(10) Pub. No.: US 2020/0071357 A1
(43) Pub. Date: Mar. 5, 2020

(54) ANTIMICROBIAL PEPTIDES

(71) Applicant: UNIVERSITETET I TROMSØ - NORGES ARKTISKE UNIVERSITET, ITromsø (NO)

(72) Inventors: Runar Gjerp SOLSTAD, Tromsø (NO); Morton B. STRØM, Tromsø (NO); Cecilie JOHANSEN, Leknes (NO); Klara STENSVÅG, Tromsø (NO); Tor HAUG, Kvaløysletta (NO)

(21) Appl. No.: 16/467,601

(22) PCT Filed: Dec. 11, 2017
(86) PCT No.: PCT/EP2017/082259

§ 371 Date: Jun. 7, 2019

(30) Foreign Application Priority Data
Dec. 9, 2016 (GB) 1620963.7

(51) Int. Cl. C07K 7/06; C07K 7/08

(52) U.S. Cl. CPC C07K 7/06; A61K 38/00; C07K 7/08

(57) ABSTRACT

The present invention relates to peptides of SEQ ID NO: 27 and peptidomimetics thereof and to their use as antimicrobial and anticancer agents. Said peptides being based on the heavy chain (HC) of a centrocin from the sea urchin Echinus esculentus.

The present invention relates to peptides and similar molecules which exhibit antimicrobial activity, in particular which exhibit antibacterial and antifungal activity.

Bacterial resistance to commercial antibiotics has increased severely over the last years. Infectious bacteria that were once easily treatable by antibiotics have now become resistant. There is consequently a pressing need to come up with alternatives to the current antimicrobial drugs. Antimicrobial peptides (AMPs), proteinaceous natural products found in all living phyla examined, may be used as drug leads to develop novel antibiotics. The AMPs may have a broad spectrum of antimicrobial activity towards both Gram-positive and Gram-negative bacteria. They have also been suggested to be less prone to resistance development in bacteria and several peptides are currently in the medical pipeline.

The centrocins are potent marine natural AMPs originally isolated and characterised from the sea urchins Strongylocentrotus droebachiensis (Li, C., et al. (2010) Dev. Comp. Immunol. 34, 959-968) and Echinus esculentus (Solstad, R. G., et al. (2016) PloS ONE 11, e0151820). Homologous genes of the peptides have also been discovered (Solstad, R. G., et al. supra) in the genome sequenced S. purpuratus (Sodergren, E., et al. (2006) Science 314, 941-952). The centrocins display antimicrobial activities against both Gram-positive and Gram-negative bacteria, as well as fungi. The centrocin AMPs (ranging from 4.4-4.8 kDa in size) have a heterodimeric structure, made up of a heavy chain (HC) of ˜30 amino acids and a light chain (LC) of ˜12 amino acids.

The present inventors have identified modified antimicrobial peptides based on the heavy chain (HC) of a centrocin from the sea urchin Echinus esculentus (EeCentrocin1), which advantageously are significantly shorter than the full-length heavy chain, yet maintain good, and preferably have improved, antimicrobial activity.

In a first aspect, the present invention provides a peptide that is 12-16 amino acids in length, wherein said peptide comprises an amino acid (AA) sequence of formula (I) (SEQ ID NO:26)

(SEQ ID NO: 26)
AA₁-AA₂-AA₃-AA₄-AA₅-AA₆-AA₇-AA₈-AA₉-AA₁₀-AA₁₁-AA₁₂ (I)

wherein
AA₁ is an amino acid that has a hydrophobicity that is less than or equal to the hydrophobicity of glycine and is not an anionic amino acid;
AA₂ and AA₃ are each an amino acid with a hydrophobic R group, said R group having at least 4 non-hydrogen atoms;
AA₄, AA₅, AA₉, AA₁₁ and AA₁₂ are each a cationic amino acid;
AA₆, AA₈ and AA₁₀ are each an amino acid that is not an anionic amino acid; and
AA₇ is an amino acid with a hydrophobic R group, said R group having at least 3 non-hydrogen atoms;
or a peptidomimetic thereof.

As described above, AA₁ is an amino acid that has a hydrophobicity (hydrophobicity value) of its R-group (side chain) that is less than or equal to the hydrophobicity (hydrophobicity value) of the glycine R-group and is not an anionic amino acid. R-group hydrophobicity (hydrophobicity values) is as determined using the method described by Abraham and Leo (Abraham, D. J. & Leo, A. J. (1987), Proteins: Struct Funct Genet, 2:130-152; reviewed in Mant C.T., et al. (2009) Biopolymers (Peptide Science) 92:573-595). Thus, whether or not a given amino acid (genetically encoded or non-genetically encoded) has a hydrophobicity that is less than or equal to the hydrophobicity of glycine is as determined using the method described by Abraham and Leo (supra). Such a determination can be readily done by a skilled person. Thus, AA₁ is an amino acid that has a hydrophobicity that is less than or equal to the hydrophobicity of glycine as determined by the method of Abraham and Leo (supra). Tables A and B below depict a hydrophobicity scale (hydrophobicity values) for genetically encoded amino acids according to the Abraham and Leo (supra) method. Table B contains the same values as Table A, but ranks the hydrophobicity of genetically encoded amino acids from highest hydrophobicity (Tryptophan, W) to lowest hydrophobicity (Glutamic acid, E).

TABLE A

| | | R- | | |
|---|---|---|---|---|
| | One- | group | R-group | |
| | letter | (side- | (mass | |
| Residue | symbol | chain) | in Da) | Hydrophobicity¹ |
| Tryptophan | W | C₉H₈N | 130 | 1.88 |
| Phenylalanine | F | C₇H₇ | 91 | 1.87 |
| Tyrosine | Y | C₇H₇O | 107 | 1.20 |
| Leucine | L | C₄H₉ | 57 | 1.81 |
| Isoleucine | I | C₄H₉ | 57 | 1.81 |
| Valine | V | C₃H₇ | 43 | 1.27 |
| Proline | P | C₃H₇N | 57 | 0.95 |
| Methionine | M | C₃H₇S | 75 | 0.81 |
| Cysteine | C | CH₃S | 47 | 0.43 |
| Alanine | A | CH₃ | 15 | 0.32 |
| Glycine | G | H | 1 | 0.00 |
| Threonine | T | C₂H₅O | 45 | −0.30 |
| Serine | S | CH₃O | 31 | −0.62 |
| Asparagine | N | C₂H₄NO | 58 | −0.97 |
| Glutamine | Q | C₃H₆NO | 72 | −1.15 |
| Aspartic acid | D | C₂H₃O₂ | 59 | −3.18 |
| Glutamic acid | E | C₃H₅O₂ | 73 | −3.84 |
| Histidine | H | C₄H₆N₂ | 82 | 0.01 |
| Lysine | K | C₄H₁₀N | 72 | −1.80 |
| Arginine | R | C₄H₁₀N₃ | 100 | −3.04 |

¹Scale: Abraham, D. J. & Leo, A. J. (1987). Proteins: Struct Funct Genet, 2: 130-152.
Calculation of amino acid side-chain partition coefficients relative to glycine by the fragment method. Calculations are based on Na-acetyl-amino-acid amide analogs. The values for Histidine, Aspartic acid, and Glutamic acid are based on the assumption that their side-chains are deprotonated.

As indicated above, the value for Histidine (H) is based on the assumption that its side-chain is deprotonated. However, in most in vivo situations it is expected that the side chain of Histidine would be partially protonated, which would make Histidine cationic, and more hydrophilic than Glycine. Thus, in accordance with the present invention, Histidine (H) is considered to be less hydrophobic than glycine and to be a cationic acid (as per its typical categorisation).

TABLE B

| Residue | Hydrophobicity |
|---|---|
| Trp | 1.88 |
| Phe | 1.87 |
| Leu | 1.81 |
| Ile | 1.81 |
| Val | 1.27 |
| Tyr | 1.20 |
| Pro | 0.95 |
| Met | 0.81 |
| Cys | 0.43 |
| Ala | 0.32 |
| His | 0.01 |
| Gly | 0.00 |
| Thr | −0.30 |
| Ser | −0.62 |
| Asn | −0.97 |
| Gln | −1.15 |
| Lys | −1.80 |
| Arg | −3.04 |
| Asp | −3.18 |
| Glu | −3.84 |

AA₁ may be a cationic amino acid or an uncharged amino acid, but is typically uncharged.

In some embodiments, AA₁ is selected from the group consisting of G, T, S, N, Q, H, K and R.

In some embodiments, AA₁ is an uncharged amino acid selected from the group consisting of G, T, S, N and Q (preferably G).

In some embodiments, AA₁ is a cationic amino, preferably lysine or arginine, but may be histidine or any non-genetically coded or modified amino acid carrying a positive charge at pH 7.0 (on its R-group/side chain). Exemplary non-genetically coded or modified amino acids are described elsewhere herein. In some embodiments, AA₁ is a cationic amino acid selected from the group consisting of H, K and R (K and R are preferred, more preferably K).

In a particularly preferred embodiment, AA₁ is Glycine (G).

As described above, AA₁ is not an anionic amino acid. Accordingly, AA₁ is not D or E.

As described above, AA₂ and AA₃ are each an amino acid with a hydrophobic R group, said R group having at least 4 non-hydrogen atoms. Preferably, said R group has at least 7 non-hydrogen atoms, more preferably at least 9 non-hydrogen atoms. Preferably, at least one of AA₂ and AA₃ has at least 7 (preferably at least 9) non-hydrogen atoms in its R group. Preferably, both AA₂ and AA₃ have R groups having at least 7 non-hydrogen atoms. More preferably, both AA₂ and AA₃ have R groups having at least 9 non-hydrogen atoms (e.g. Tryptophan, W).

In some embodiments, AA₂ and AA₃ are each an amino acid with a hydrophobic R group, said R group having 4-27 non-hydrogen atoms, preferably, 7-27, more preferably 9-27 non-hydrogen atoms. In preferred embodiments, the R group contains 1 or more (e.g. 1, 2 or 3) cyclic groups which will typically comprise 5 or 6 non-hydrogen atoms (preferably 6 non-hydrogen atoms). If two or more cyclic groups are present, these are typically fused or connected. In the case of fused rings of course the non-hydrogen atoms can be shared.

In preferred embodiments, one or both (preferably both) of AA₂ and AA₃ has a hydrophobic R group that has a mass of >90Da.

In AA₂ and AA₃, the hydrophobic R group may contain hetero atoms such as O, N or S but typically there is no more than one heteroatom, preferably it is nitrogen. This R group will preferably have no more than 2 polar groups, more preferably none or one, most preferably none.

In some embodiments, AA₂ and AA₃ are each independently selected from the group consisting of W, F, Y, L and I. Preferably, AA₂ and AA₃ are each independently selected from the group consisting of W, F and Y.

In preferred embodiments, at least one of AA₂ and AA₃ is W, F or Y, preferably both AA₂ and AA₃ are W, F or Y.

In particularly preferred embodiments, at least one of AA₂ and AA₃ is W (AA₂ is W and/or AA₃ is W), preferably both AA₂ and AA₃ are W.

Typically, at least one (preferably both) of AA₂ and AA₃ are genetically encoded amino acids, for example as described above. However, one or both of AA₂ and AA₃ may be a non-genetically encoded amino acid. For example, AA₂ and AA₃ (one or both) may be tributyl tryptophan (Tbt), biphenylalanine (Bip) or a biphenylalanine derivative such as Bip (4-(2-Naphthyl)), Bip (4-(1-Naphthyl)), Bip (4-n-Bu), Bip (4-Ph) or Bip (4-T-Bu); Bip (4-(2-Naphthyl)).

AA₂ and AA₃typically have an R-group (side chain) that is at least as hydrophobic as the leucine or isoleucine R-groups or at least as hydrophobic as the tyrosine R-group, particularly preferably at least as hydrophobic as the phenylalanine or tryptophan (preferably tryptophan) R-groups, as determined by the method of Abraham and Leo (supra).

As described above, AA₄, AA₅, AA₉, AA₁₁ and AA₁₂ are each a cationic amino acid. Preferably, AA₄, AA₅, AA₉, AA₁₁ and AA₁₂ are each, independently, lysine (K) or arginine (R) but may be histidine (H) or any non-genetically coded or modified amino acid carrying a positive charge at pH 7.0 (on its R-group/side chain).

In some embodiments, AA₄, AA₅, AA₉, AA₁₁ and AA₁₂ are each independently selected from the group consisting of K, R and H, preferably R and K.

In some preferred embodiments AA₄ is R. In some preferred embodiments AA₅ is R. In some preferred embodiments AA₉ is K. In some preferred embodiments AA₁₁ is R. In some preferred embodiments AA₁₂ is K.

In a particularly preferred embodiment, AA₄ is R, AA₅ is R, AA₉ is K, AA₁₁ is R and AA₁₂ is K.

Typically, at least one (preferably at least 2, or at least 3, or at least 4, more preferably all) of AA₄, AA₅, AA₉, AA₁₁ and AA₁₂ are genetically encoded amino acids, for example as described above. However, one or more of AA₄, AA₅, AA₉, AA₁₁ and AA₁₂ may be a non-genetically encoded cationic amino acid. Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

As described above, AA₆, AA₈ and AA₁₀ are each an amino acid that is not an anionic amino acid. Anionic amino acids carry a negative charge at pH 7.0 (on the R-group/side chain). Put another way, AA₆, AA₈ and AA₁₀ are not acidic amino acids. Accordingly, AA₆, AA₈ and AA₁₀ are not D or E.

One or more (preferably two, more preferably all) of AA₆, AA₈ and AA₁₀, are amino acids may be in accordance with the definitions of AA₁, AA₂, AA₃, AA₄, AA₅, AA₇, AA₉, AA₁₁ or AA₁₂.

Preferably, one or more (preferably two, more preferably all) of AA₆, AA₈ and AA₁₀ is an uncharged amino acid (e.g. W, F, Y, L, I, V, P, M, C, A, G, T, S, N or Q).

In some embodiments, one or more (1, 2 or 3) of AA₆, AA₈ and AA₁₀ is a cationic amino acid. Suitable cationic acids are described herein in connection with AA₄, AA₅, AA₉, AA₁₁ and AA₁₂.

In some embodiments, AA₆, AA₈ and AA₁₀, are each independently selected from the group consisting of W, F, Y, L, I, V, P, M, C, A, G, T, S, N, Q, H, K and R. In some embodiments, AA₆, AA₈ and AA₁₀, are each independently selected from the group consisting of W, F, Y, L, I, V, M, A, G, T, S, N, Q, H, K and R.

In some embodiments, AA₆ is an amino acid in accordance with the definitions of AA₁, AA₂, AA₃, AA₄, AA₅, AA₇, AA₉, AA₁₁ or AA₁₂.

In some embodiments, AA₆ is an amino acid that has an R-group with a hydrophobicity that is less than or equal to the hydrophobicity of the glycine R-group (e.g. T, S, N, Q, H, K or R). Thus, the description of the AA₁ residues above may be applied mutatis mutandis to AA₆. In other embodiments, AA₆ is an amino acid that has an R-group with a hydrophobicity that is greater than or equal to the hydrophobicity of the glycine R-group (e.g. W or A, preferably W).

In some embodiments, AA₆is an amino acid with a hydrophobic R group, said R group having at least 3 non-hydrogen atoms, (e.g. W, F, Y, L, I, V, P or M) or 4 non-hydrogen atoms (e.g. W, F, Y, L or I), or at least 7 non-hydrogen atoms (e.g. W, F or Y), or at least 9 non-hydrogen atoms (e.g. W). Thus, the description of the AA₂, AA₃ or AA₇ residues above may be applied mutatis mutandis to AA₆.

In some embodiments, AA₆is a cationic amino acid. Thus, the description of the AA₄, AA₅, AA₉, AA₁₁ and AA₁₂ residues above may be applied mutatis mutandis to AA₆. In some embodiments, AA₆ is K.

In preferred embodiments, AA₆ is uncharged. In some embodiments, AA₆ is T, S, N, Q, W or A. Preferably, AA₆ is T, S, N or Q or W (or T, S, N or Q), more preferably T or S. In some embodiments, AA₆ is T, A or W, preferably T or W. In particularly preferred embodiments, AA₆ is T. In other preferred embodiments, AA₆ is W.

In some preferred embodiments, AA₆ is an amino acid that has a hydrophobicity that is greater than or equal to the hydrophobicity of threonine. This can be determined using the method described by Abraham and Leo (supra).

In some embodiments, AA₈ is an amino acid in accordance with the definitions of AA₁, AA₂, AA₃, AA₄, AA₅, AA₇, AA₉, AA₁₁ or AA₁₂.

In some embodiments, AA₈ may be an amino acid that has an R-group with a hydrophobicity that is less than or equal to the hydrophobicity of the glycine R-group (e.g. T, S, N, Q, H, K or R). Thus, the description of the AA₁ residues above may be applied mutatis mutandis to AA₈. However, in some embodiments, AA₈ may be an amino acid that has an R-group with a hydrophobicity that is greater than or equal to the hydrophobicity of the glycine R-group (e.g. A).

In some embodiments, AA₈is a cationic amino acid. Thus, the description of the AA₄, AA₅, AA₉, AA₁₁ and AA₁₂ residues above may be applied mutatis mutandis to AA₈. In some embodiments, AA₈ is K.

In some embodiments, AA₈ is uncharged. In some embodiments, AA₈is T, S, N, or Q or A. In a particularly preferred embodiment, AA₈ is A.

In some preferred embodiments, AA₈ is an amino acid that has a hydrophobicity that is less than or equal to the hydrophobicity of alanine and is not an anionic amino acid. This can be determined using the method described by Abraham and Leo (supra).

In some embodiments, AA₁₀ is an amino acid in accordance with the definitions of AA₁, AA₂, AA₃, AA₄, AA₅, AA₇, AA₉, AA₁₁ or AA₁₂.

In some embodiments, AA₁₀ may be an amino acid that has an R-group with a hydrophobicity that is less than or equal to the hydrophobicity of the glycine R-group (e.g. T, S, N, Q, H, K or R). Thus, the description of the AA₁ residues above may be applied mutatis mutandis to AA₁₀. However, preferably, AA₁₀ is an amino acid that has an R-group with a hydrophobicity that is greater than or equal to the hydrophobicity of the glycine R-group (e.g. V). Preferably, AA₁₀ is not Alanine (A).

Indeed, preferably, AA₁₀ is an amino acid with a hydrophobic R group, said R group having at least 3 non-hydrogen atoms, (e.g. W, F, Y, L or I, V, P or M) or 4 non-hydrogen atoms (e.g. W, F, Y, L or I), or at least 7 non-hydrogen atoms (e.g. W, F or Y), or at least 9 non-hydrogen atoms (e.g. W). Thus, the description of the AA₇ residues elsewhere herein may be applied mutatis mutandis to AA₁₀. The description of the AA₂ and AA₃ residues elsewhere herein may be applied mutatis mutandis to AA₁₀.

Thus, in some embodiments, AA₁₀ is selected from the group consisting of W, F, Y, L or I, V, P or M (preferably W, F, Y, L, I, or V). In preferred embodiments, AA₁₀ is V.

In other embodiments, AA₁₀ is a cationic amino acid. Thus, the description of the AA₄, AA₅, AA₉, AA₁₁ and AA₁₂ residues above may be applied mutatis mutandis to AA₁₀.

In some embodiments, AA₁₀ is uncharged.

In some embodiments, AA₆ is T, A or W (preferably T or W) and/or AA₈ is A or K or R, preferably A or K (preferably A) and/or AA₁₀ is V. In some embodiments, AA₆ is T and AA₈ is A and AA₁₀ is V. In some embodiments, AA₆ is W, AA₈ is A and AA₁₀ is V.

As described above, AA₇ is an amino acid with a hydrophobic R group, said R group having at least 3 non-hydrogen atoms (e.g. W, F, Y, L, I, V, P or M). In some embodiments, said R group has at least 4 non-hydrogen atoms (e.g. W, F, Y, L, I, P or M) or at least 7 non-hydrogen atoms (e.g. W, F or Y) or at least 9 non-hydrogen atoms (e.g. W). Thus, in some embodiments, AA₇ is an amino acid in accordance with the definition of AA₂ and AA₃ elsewhere herein.

In some embodiments, AA₇ is an amino acid with a hydrophobic R group, said R group having 3-27 (or 4-27 or 7-27 or 9-27) non-hydrogen atoms. In some embodiments, the R group may contain 1 or more (e.g. 1, 2 or 3) cyclic groups as described elsewhere herein in connection with AA₂ and AA₃.

In AA₇, the hydrophobic R group may contain hetero atoms such as O, N or S but typically there is no heteroatom or no more than one heteroatom (preferably it is nitrogen). This R group will preferably have no more than 2 polar groups, more preferably none or one, most preferably none.

In some embodiments, AA₇ is selected from the group consisting of W, F, Y, L, I, V, P and M. In some embodiments, AA₇ is selected from the group consisting of W, F, Y, L, I and V. In preferred embodiments, AA₇ is V.

Typically, AA₇ is a genetically encoded amino acid, for example as described above. However, AA₇ may be a non-genetically encoded amino acid, e.g. as described elsewhere herein in connection with AA₂ and AA₃.

AA₇typically has an R-group that is at least as hydrophobic as the valine R-group, as determined by the method of Abraham and Leo (supra). AA₇ may have an R-group that is at least as hydrophobic as the leucine or isoleucine or tyrosine R-groups, or may have an R-group that is at least as hydrophobic as the phenylalanine or tryptophan R-groups.

In some preferred embodiments, the peptide (or peptidomimetic) does not contain any anionic amino acid residues.

In a preferred embodiment, the present invention provides a peptide that is 12-16 amino acids in length (preferably 12 amino acids in length), wherein said peptide comprises (or consists of) the amino acid sequence GWWRRTVAKVRK (SEQ ID NO:10), or a peptidomimetic thereof. Preferably, such molecules are amidated at the C-terminus.

In a particularly preferred embodiment, the present invention provides a peptide that is 12 amino acids in length, wherein said peptide consists of the amino acid sequence GWWRRTVAKVRK (SEQ ID NO:10). Preferably, said peptide is amidated at its C-terminus.

In another embodiment, the present invention provides a peptide that is 12-16 amino acids in length (preferably 12 amino acids in length), wherein said peptide comprises (or consists of) the amino acid sequence GWWRRWVAKVRK (SEQ ID NO:20), or a peptidomimetic thereof. Preferably, such molecules are amidated at the C-terminus.

In one embodiment, the present invention provides a peptide that is 12 amino acids in length, wherein said peptide consists of the amino acid sequence GWWRRWVAKVRK (SEQ ID NO:20). Preferably, said peptide is amidated at its C-terminus.

In another embodiment, the present invention provides a peptide that is 12-16 amino acids in length (preferably 12 amino acids in length), wherein said peptide comprises (or consists of) the amino acid sequence GWWRRKVAKVRK (SEQ ID NO:21), or a peptidomimetic thereof. Preferably, such molecules are amidated at the C-terminus.

In another embodiment, the present invention provides a peptide that is 12-16 amino acids in length (preferably 12 amino acids in length), wherein said peptide comprises (or consists of) the amino acid sequence GWWRRWVKKVRK (SEQ ID NO:22), or a peptidomimetic thereof. Preferably, such molecules are amidated at the C-terminus.

In another embodiment, the present invention provides a peptide that is 12-16 amino acids in length (preferably 12 amino acids in length), wherein said peptide comprises (or consists of) the amino acid sequence KWWRRWVKKVRK (SEQ ID NO:23), or a peptidomimetic thereof. Preferably, such molecules are amidated at the C-terminus.

In another embodiment, the present invention provides a peptide that is 12-16 amino acids in length (preferably 12 amino acids in length), wherein said peptide comprises (or consists of) the amino acid sequence RWWRRWVRRVRR (SEQ ID NO:25), or a peptidomimetic thereof. Preferably, such molecules are amidated at the C-terminus.

In another embodiment, the present invention provides a peptide that is 12-16 amino acids in length (preferably 12 amino acids in length), wherein said peptide comprises (or consists of) an amino acid sequence selected from the group consisting of SEQ ID NO:10, SEQ ID NO:8, SEQ ID NO:20, SEQ ID NO:22, SEQ ID NO:23 and SEQ ID NO:25, or a peptidomimetic thereof. Preferably, such molecules are amidated at the C-terminus.

In another embodiment, the present invention provides a peptide that is 12-16 amino acids in length (preferably 12 amino acids in length), wherein said peptide comprises (or consists of) the amino acid sequence GWWRRAVAKVRK (SEQ ID NO:8), or a peptidomimetic thereof. Preferably, such molecules are amidated at the C-terminus.

In one embodiment, the present invention provides a peptide that is 12-16 amino acids in length (preferably 12 amino acids in length), wherein said peptide comprises (or consists of) the amino acid sequence GWWRRTVAKVRK (SEQ ID NO:10), or a sequence substantially homologous thereto, wherein said substantially homologous sequence contains 1, 2 or 3 amino acid substitutions (amino acid replacements) compared to the given amino acid sequence (SEQ ID NO:10), and wherein
(i) if the G at position 1 is replaced, the replacement amino acid is in accordance with AA₇ as defined elsewhere herein;
(ii) if one or both of the W residues at positions 2 and 3 is replaced, the replacement amino acid is in accordance with AA₂ or AA₃ as defined elsewhere herein;
(iii) if one or more of the residues at positions 4 (i.e. R), 5 (i.e. R), 9 (i.e. K), 11 (i.e. R) and 12 (i.e. K) is replaced, the replacement amino acid is in accordance with AA₄, AA₅, AA₉, AA₁₁ or AA₁₂ as defined elsewhere herein;
(iv) if one or more of the residues at positions 6 (i.e. T), 8 (i.e. A) or 10 (i.e. V) is replaced, the replacement amino acid is in accordance with AA₆, AA₈or AA₁₀, as defined elsewhere herein; and
(v) if the V at position 7 is replaced, the replacement amino acid is in accordance with AA₇ as defined elsewhere herein.

Preferably, such molecules are amidated at the C-terminus. Peptidomimetic versions of such peptides are also provided.

In another aspect, the present invention provides a peptide that is 12-16 amino acids in length, wherein said peptide comprises an amino acid (AA) sequence of formula (IB) SEQ ID NO:28

(IB)
SEQ ID NO: 28
AA₁-AA₂-AA₃-AA₄-AA₅-AA₆-AA₇-AA₈-AA₉-AA₁₀-AA₁₁-AA₁₂

wherein
AA₁ is an amino acid that has a hydrophobicity that is less than or equal to the hydrophobicity of glycine and is not an anionic amino acid;
AA₂ and AA₃ are each an amino acid with a hydrophobic R group, said R group having at least 4 non-hydrogen atoms;
AA₄, AA₅, AA₉, AA₁₁ and AA₁₂ are each a cationic amino acid;
AA₆ and AA₈ are each an amino acid that is not an anionic amino acid; and
AA₇ and AA₁₀ are each an amino acid with a hydrophobic R group, said R group having at least 3 non-hydrogen atoms;
or a peptidomimetic thereof.

Other features and properties of other aspects of the invention apply, mutatis mutandis, to this aspect of the invention (i.e. to the formula (IB)-based aspect of the invention).

In another aspect, the present invention provides a peptide that is 12-16 amino acids in length (preferably 12 amino acids in length), wherein said peptide comprises (or consists of) the amino acid sequence GWARRWVAKVRK (SEQ ID NO:24), or a peptidomimetic thereof. Preferably, such molecules are amidated at the C-terminus.

Peptides comprising (or consisting of) the amino acid sequences set forth in SEQ ID NOs 21 and 24 are typically not preferred.

In a preferred aspect, the present invention provides a peptide that is 12-16 amino acids in length, wherein said peptide comprises an amino acid (AA) sequence of formula (IA) (SEQ ID NO:27)

(IA)
(SEQ ID NO: 27)
AA₁-AA₂-AA₃-AA₄-AA₅-AA₆-AA₇-AA₈-AA₉-AA₁₀-AA₁₁-AA₁₂

wherein
AA₁ is an amino acid that has a hydrophobicity that is less than or equal to the hydrophobicity of glycine and is not an anionic amino acid;
AA₂ and AA₃ are each an amino acid with a hydrophobic R group, said R group having at least 4 non-hydrogen atoms;
AA₄, AA₅, AA₉, AA₁₁ and AA₁₂ are each a cationic amino acid;
AA₆ is an uncharged amino acid;
AA₈ and AA₁₀ are each an amino acid that is not an anionic amino acid; and
AA₇ is an amino acid with a hydrophobic R group, said R group having at least 3 non-hydrogen atoms;
or a peptidomimetic thereof.

Other features and properties of other aspects of the invention apply, mutatis mutandis, to this aspect of the invention (i.e. to the formula (IA)-based aspect of the invention).

As is evident from elsewhere herein, amino acids may be genetically encoded or non-genetically encoded. Genetically encoded amino acids are typically preferred.

As described elsewhere herein, preferred peptides of the invention are 12-16 (preferably 12) amino acids in length. However, in another aspect, peptides of the invention may be 8-16 amino acids in length (e.g. 8-11 or 8-12 amino acids in length). In such an aspect, if the peptide is less than 12 amino acids in length (8, 9, 10 or 11 amino acids in length), 1, 2, 3 or 4 (preferably 2, more preferably 1) amino acids of the peptide of formula (I) or (IA) or (IB) will not be present in the peptide. Thus, in one aspect, the present invention provides a peptide that is 8-11 amino acids in length (preferably 10 or 11 amino acids in length), wherein said peptide is a peptide based on the peptide of formula (I) or (IA) or (IB) as described above and wherein 1, 2, 3, or 4 (preferably 2, more preferably 1) of the amino acids (AA) of the peptide of formula (I) or (IA) or (IB) are absent (deleted or removed). Peptidomimetics of such peptides are also provided by the present invention. Other features and properties of other aspects of the invention apply, mutatis mutandis, to this aspect of the invention.

As described elsewhere herein, molecules of the invention have antimicrobial activity.

Preferably, molecules of the invention have a minimal inhibitory concentration (MIC) against Gram positive bacteria of 25 μM or less, preferably 12.5 μM or less. Preferably, molecules of the invention have a MIC against Corynebacterium glutamicum (e.g. ATCC 13032) and against Staphylococcus aureus (e.g. ATCC 9144) of 25 μM or less, preferably 12.5 μM or less. Preferably, molecules of the invention have a MIC against Corynebacterium glutamicum (e.g. ATCC 13032) of 1.6 μM or less, preferably 0.8 μM or less, preferably 0.5 μM or less, more preferably 0.4 μM or less. Preferably, molecules of the invention have a MIC against Staphylococcus aureus (e.g. ATCC 9144) of 25 μM or less, preferably 20 μM or less, preferably 15 μM or less, more preferably 12.5 μM or less.

Preferably, molecules of the invention have a minimal inhibitory concentration (MIC) against Gram negative bacteria of 10 μM or less, preferably 5 μM or less, preferably 4 μM or less or 3.1 μM or less. Preferably, molecules of the invention have a minimal inhibitory concentration (MIC) against Pseudomonas aeruginosa (e.g. ATCC 27853) and against Escherichia coli (e.g. ATCC 25922) of 10 μM or less, 5 μM or less, preferably 4 μM or less or 3.1 μM or less. Preferably, molecules of the invention have a MIC against Pseudomonas aeruginosa (e.g. ATCC 27853) of 10 μM or less, 5 μM or less, preferably 4 μM or less or 3.1 μM or less, more preferably 2 μM or 1.6 μM or less. Preferably, molecules of the invention have a MIC against Escherichia coli (e.g. ATCC 25922) of 10 μM or less, 5 μM or less, preferably 4 μM or less or 3.1 μM or less.

Suitable assays to determine the MIC of molecules of the invention against bacteria are known in the art. A particularly suitable and preferred assay is described in the Example section herein. Minimum inhibitory concentration (MIC) may be defined as the lowest concentration showing complete inhibition of bacterial growth (e.g. in a 24h incubation period, e.g. at 35° C.) (e.g. as measured by optical density at 595 nm).

Preferably, molecules of the invention have a minimal inhibitory concentration (MIC) against fungi of 50 μM or less or 25 μM or less, preferably 12.5 μM or less, preferably 10 μM or less, more preferably 6.3 μM or less.

Preferably, molecules of the invention have a minimal inhibitory concentration (MIC) against Candida albicans (e.g. ATCC 10231) and against Aureobasidium pullulans and against Rhodotorula sp. of 50 μM or less or 25 μM or less, preferably 12.5 μM or less, preferably 10 μM or less, more preferably 6.3 μM or less. Preferably, molecules of the invention have a MIC against Candida albicans (e.g. ATCC 10231) of 25 μM or less,12.5 μM or less, preferably 10 μM or less or 5 μM or less, more preferably 3.1 μM or less. Preferably, molecules of the invention have a MIC against Aureobasidium pullulans of 50 μM or less or 25 μM or less, preferably 12.5 μM or less, preferably 10 μM or less, more preferably 6.3 μM or less. Preferably, molecules of the invention have a MIC against Rhodotorula sp. of 12.5 μM or less, 10 μM or less, preferably 5 μM or less or 2 μM or less, more preferably 1.6 μM or less.

Suitable assays to determine the MIC of molecules of the invention against fungi are known in the art. A particularly suitable and preferred assay is described in the Example section herein. Minimum inhibitory concentration (MIC) may be defined as the lowest concentration giving no visible fungal growth (e.g. as determined visually, e.g. after incubation for 24 h, e.g. at room temperature).

Preferably, molecules of the invention have good antimicrobial activity against bacteria and fungi. For example, molecules of the invention may have a MIC of 50 μM or less or 25 μM or less, preferably 12.5 μM or less, against each one of Corynebacterium glutamicum (e.g. ATCC 13032), Staphylococcus aureus (e.g. ATCC 9144), Pseudomonas aeruginosa (e.g. ATCC 27853), Escherichia coli (e.g. ATCC 25922), Candida albicans (e.g. ATCC 10231), Aureobasidium pullulans and Rhodotorula sp. (i.e. have a MIC of 50 μM or less or 25 μM or less, preferably 12.5 μM or less, against all of these bacterial and fungal species).

The molecules of the invention are significantly shorter than the full-length EeCentrocin 1 heavy chain (HC) (SEQ ID NO:1). Molecules of the invention may be 8-16 amino acids in length (8, 9, 10, 11, 12, 13, 14, 15 or 16 amino acids in length). In some embodiments, molecules of the invention may be 8-11 amino acids in length (8, 9, 10 or 11 amino acids in length). In preferred embodiments, molecules of the invention are 12-16 amino acids in length (12, 13, 14, 15 or 16 amino acids in length). Particularly preferred molecules of the invention are 12 amino acids in length. Without wishing to be bound by theory, shorter molecules may have certain advantages over longer molecules including, for example, that they are easier and cheaper to synthesise, they may have decreased immunogenicity and they may have better penetration into microbial populations (e.g. in biofilms or mucus). Shorter peptides may also give a higher yield after synthesis, be easier to purify, be easier to dissolve and/or be easier to administer.

Peptides are preferred molecules of the invention. Molecules of the invention are typically linear peptides or peptidomimetics. However, in some embodiments, the peptides or peptidomimetics may be cyclic. For example, in some embodiments the N-terminus and the C-terminus of the peptide or peptidomimetic are linked with a covalent bond that generates a ring. Methods for the cyclisation of peptides are known in the art.

In certain preferred embodiments, the peptides (or peptidomimetics) of the present invention are amidated at the C-terminus (i.e. the C-terminal amino acid residue may be amidated). Methods of amidating the C-terminal amino acid of peptides are known in the art. Without wishing to be bound by theory it is believed that C-terminal amidation of the molecules of the invention may be advantageous as it neutralizes negative charge created by the C-terminal COOH group. Peptides (or peptidomimetics) of the present invention may be, but typically are not, esterified at the C-terminus (i.e. the C-terminal amino acid residue may modified with an ester). Methods of C-terminal esterification of peptides are known in the art.

β and γ amino acids as well as a amino acids are included within the term ‘amino acids’, as are N-substituted glycines which may all be considered AA units. α amino acids are generally preferred. The molecules of the invention include beta peptides and depsipeptides.

The molecules of the present invention may be peptidomimetics and peptidomimetics of the peptides described and defined herein are a further aspect of the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the molecule may be reacting with a membrane rather than the specific active site of an enzyme, some of the problems described of exactly mimicing affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46,47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The term ‘amino acid’ may conveniently be used herein to refer to the equivalent sub-units of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA(1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C terminii which may be modified as discussed herein.

The peptides of the invention may be synthesised in any convenient way. Generally the reactive groups present (for example amino, thiol and/or carboxyl) will be protected during overall synthesis. The final step in the synthesis will generally be the deprotection of a protected derivative of the invention.

In building up the peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art. A microwave assisted Fmoc-based solid phase peptide synthesis may be used, e.g. described in the Example section herein.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the a-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPCIP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed. The side chain protecting groups must be stable to the conditions used to remove the temporary a-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

References and techniques for synthesising peptidomimetic compounds and the other bioactive molecules of the invention are described herein and thus are well known in the art.

Typically, compounds (peptides or peptidomimetics) of the present invention have low or negligible haemolytic activity when used at (or close to) their minimal inhibitory concentration. Haemolytic activity may be as assessed against human red blood cells, for example using the haemolytic activity assay described in the Example section herein.

Typically, compounds of the present invention have lower (preferably significantly lower) haemolytic activity than the full-length EeCentrocin 1 HC (SEQ ID NO:1). For example, in some embodiments, compounds of the present invention exhibit haemolytic activity (e.g. against human red blood cells) that is 50% or less (preferably 40% or less, or 30% or less, more preferably 20% or less) of the haemolytic activity exhibited by the full-length EeCentrocin 1 HC (SEQ ID NO:1) when used at a concentration of 25 μM.

As described elsewhere herein, molecules (peptides and peptidomimetics) of the present invention exhibit antimicrobial activity. Without wishing to be bound by theory, it is believed that the molecules of the present invention may exert a cytotoxic effect through a direct membrane-affecting mechanism and thus may be termed membrane acting antimicrobial agents. These molecules may be lytic, destabilising or even perforating the cell membrane. This may offer a distinct therapeutic advantage over agents which act on or interact with proteinaceous components of the target cells, e.g. cell surface receptors. While mutations may result in new forms of the target proteins leading to antibiotic resistance, it is much less likely that radical changes to the lipid membranes could occur to prevent the cytotoxic effect. A lytic effect may cause very rapid cell death and thus has the advantage of killing bacteria before they have a chance to multiply. Again, without wishing to be bound by theory, it is believed that molecules of the invention may be attracted to the negatively charged phospholipids of the cell membrane by virtue of the presence of cationic residues, and that hydrophobic groups may be able to destabilise the normal three dimensional lipid bi-layer configuration of microbial (e.g. bacterial or fungal) cell membranes. This interaction may increase permeability and result in a loss of membrane integrity and eventually cell lysis and death.

Thus in a further aspect is provided the molecules of the invention for use in destabilising and/or permeabilising microbial cell membranes. By ‘destabilising’ is meant a perturbation of the normal three dimensional lipid bi-layer configuration including but not limited to membrane thinning, increased membrane permeability (typically not involving channels) to water, ions or metabolites etc. which also impairs the respiratory systems of the bacteria.

In a further aspect the present invention provides the peptides or peptidomimetics defined herein (or compositions or formulations comprising such molecules) for use in therapy, in particular for use in the treatment of microbial infections (e.g. a bacterial and/or fungal infection). Thus, in one aspect, the present invention provides the peptides or peptidomimetics defined herein for use in the treatment of a bacterial infection. In another aspect, the present invention also provides the peptides or peptidomimetics defined herein for use in the treatment of a fungal infection.

Preferred molecules of the invention are active both as antibacterial agents and antifungal agents.

Treatment includes prophylactic treatment.

Alternatively viewed, the peptides or peptidomimetics defined herein are for use as an antimicrobial agent (e.g. antibacterial or antifungal agent).

Alternatively viewed the present invention provides a method of treating a microbial infection (e.g. a bacterial and/or fungal infection) which method comprises administering to a patient in need thereof a therapeutically effective amount of a peptide or peptidomimetic of the invention as defined herein.

A therapeutically effective amount will be determined based on the clinical assessment and can be readily monitored. Typically, the amount administered should be effective to kill all or a proportion of the target microbes or to prevent or reduce their rate of reproduction or otherwise to lessen their harmful effect on the body. The clinician or patient should observe improvement in one or more of the parameters or symptoms associated with the infection.

Further alternatively viewed, the present invention provides the use of a peptide or peptidomimetic of the invention as defined herein in the manufacture of a medicament for treating a microbial infection (e.g. a bacterial and/or fungal infection).

As mentioned above, compounds of the present invention may be used in the treatment of a bacterial infection. Such infections include infections with Gram positive (G+) bacteria or Gram negative (G-) bacteria. For example, compounds of the present invention may be used in the treatment of an Escherichia coli (Ec) infection, a Pseudomonas aeruginosa (Pa) infection, a Staphylococcus aureus (Sa) infection and/or a Corynebacterium glutamicum (Cg) infection. Compounds of the present invention may also be used in the treatment of a Staphylococcus epidermidis infection.

As mentioned above, compounds of the present invention may be used in the treatment of a fungal infection. For example, compounds of the present invention may be used in the treatment of Candida albicans (Ca) infection, a Rhodotorula sp. (Rh) infection and/or an Aureobasidium pullulans (Ap) infection. Compounds of the present invention may be used in the treatment of a yeast infection.

These treatments may involve co-administration with another antimicrobial agent.

Compounds of the present invention may also have anti-cancer (e.g. anti-tumour) activity. Accordingly, in some embodiments, the invention provides a compound (peptide or peptidomimetic) of the present invention for use in the treatment of cancer (e.g. in the treatment of tumours such as solid tumours). Thus, compounds of the invention may be used as antitumoural agents. Alternatively viewed, the present invention provides a method of treating cancer (e.g. a tumour) which method comprises administering to a patient in need thereof a therapeutically effective amount of a peptide or peptidomimetic of the invention as defined herein. Further alternatively viewed, the present invention provides the use of a peptide or peptidomimetic of the invention as defined herein in the manufacture of a medicament for treating cancer (e.g. a tumour).

The antimicrobial medical uses and methods described herein may, in preferred embodiments, be for use in patients with cystic fibrosis.

Subjects treated in accordance with the present invention will preferably be humans but veterinary treatments are also contemplated.

Such antimicrobial molecules also have non-therapeutic uses (ex vivo uses), for example in agriculture or in domestic or industrial situations as sterilising agents for materials susceptible to microbial contamination. Thus, in a further aspect, the present invention provides the use of the molecules of the invention as antimicrobial agents, particularly as antibacterial and/or antifungal agents. Methods of treating environmental or agricultural sites or products, as well as foodstuffs and sites of food production, or surfaces or tools e.g. in a hospital environment with one or more of the molecules of the invention to reduce the numbers of viable bacteria present or limit bacterial growth or reproduction constitute further aspects of the present invention.

Molecules of the present invention may also have anti-fouling, anti-biofilm (e.g. against bacterial or fungal biofilms) and/or antiparasitic uses. Thus, molecules of the present invention may be used as anti-fouling agents, anti-biofilm agents (e.g. against bacterial or fungal biofilms) and/or antiparasitic agents. Accordingly, the invention provides molecules (peptides or peptidomimetics) as defined herein for use in treating a bacterial (e.g. Staphylococcus epidermidis) or fungal infection, wherein said bacterial or fungal infection is in the form of a biofilm. The invention also provides molecules (peptides or peptidomimetics) as defined herein for use in treating a parasitic infection.

Formulations comprising one or more compounds of the invention in admixture with a suitable diluent, carrier or excipient constitute a further aspect of the present invention. Such formulations may be for, inter alia, pharmaceutical (including veterinary) purposes. Suitable diluents, excipients and carriers are known to the skilled man.

The compositions (formulations), e.g. pharmaceutical compositions, according to the invention may be presented, for example, in a form suitable for oral, nasal, parenteral, intravenal, topical or rectal administration.

As used herein, the term “pharmaceutical” includes veterinary applications of the invention.

The active compounds defined herein may be presented in the conventional pharmacological forms of administration, such as tablets, coated tablets, nasal sprays, solutions, emulsions, liposomes, powders, capsules or sustained release forms.

Formulations for topical administration are preferably in the form of a gel, cream, lotion, paste or other preparation which is more viscous than water. Further formulations for topical application include dressings, gauzes etc. which have been impregnated with a compound of the invention; when impregnating such materials the preparation containing a compound of the invention need not be more viscous than water. Conventional pharmaceutical excipients as well as the usual methods of production may be employed for the preparation of these forms.

Tablets may be produced, for example, by mixing the active ingredient or ingredients with known excipients, such as for example with diluents, such as calcium carbonate, calcium phosphate or lactose, disintegrants such as corn starch or alginic acid, binders such as starch or gelatin, lubricants such as magnesium stearate or talcum, and/or agents for obtaining sustained release, such as carboxypolymethylene, carboxymethyl cellulose, cellulose acetate phthalate, or polyvinylacetate.

The tablets may if desired consist of several layers. Coated tablets may be produced by coating cores, obtained in a similar manner to the tablets, with agents commonly used for tablet coatings, for example, polyvinyl pyrrolidone or shellac, gum arabic, talcum, titanium dioxide or sugar. In order to obtain sustained release or to avoid incompatibilities, the core may consist of several layers too. The tablet-coat may also consist of several layers in order to obtain sustained release, in which case the excipients mentioned above for tablets may be used.

Organ specific carrier systems may also be used.

Injection solutions may, for example, be produced in the conventional manner, such as by the addition of preservation agents, such as p-hydroxybenzoates, or stabilizers, such as EDTA. The solutions are then filled into injection vials or ampoules.

Nasal sprays may be formulated similarly in aqueous solution and packed into spray containers either with an aerosol propellant or provided with means for manual compression. Capsules containing one or several active ingredients may be produced, for example, by mixing the active ingredients with inert carriers, such as lactose or sorbitol, and filling the mixture into gelatin capsules.

Suitable suppositories may, for example, be produced by mixing the active ingredient or active ingredient combinations with the conventional carriers envisaged for this purpose, such as natural fats or polyethyleneglycol or derivatives thereof.

Dosage units containing the active molecules preferably contain 0.1-10 mg, for example 1-5 mg of the antimicrobial agent. The pharmaceutical compositions may additionally comprise further active ingredients, including other cytotoxic agents such as other antimicrobial peptides. Other active ingredients may include different types of antibiotics.

The bioactive molecules, when used in topical compositions, are generally present in an amount of at least 0.1%, by weight. In most cases, it is not necessary to employ the peptide in an amount greater than 1.0%, by weight.

In employing such compositions systemically (intra-muscular, intravenous, intraperitoneal), the active molecule may be present in an amount to achieve a serum level of the bioactive molecule of at least about 5 pg/ml. In general, the serum level need not exceed 500 pg/ml. A preferred serum level is about 100 ug/ml. Such serum levels may be achieved by incorporating the bioactive molecule in a composition to be administered systemically at a dose of from 1 to about 10 mg/kg. In general, the molecule(s) need not be administered at a dose exceeding 100 mg/kg.

AMINO ACID SEQUENCES DISCLOSED HEREIN AND
THEIR SEQUENCE IDENTIFIERS (SEQ ID NOs)
(full-length heavy chain of EeCentrocin 1)
SEQ ID NO: 1
GWWRRTVDKVRNAGRKVAGFASKACGALGH
(full-length light chain of EeCentrocin 1)
SEQ ID NO: 2
DIGKYCGYAHALN
(G1A)
SEQ ID NO: 3
AWWRRTVAKVRK
(W2A)
SEQ ID NO: 4
GAWRRTVAKVRK
(W3A)
SEQ ID NO: 5
GWARRTVAKVRK
(R4A)
SEQ ID NO: 6
GWWARTVAKVRK
(R5A)
SEQ ID NO: 7
GWWRATVAKVRK
(T6A)
SEQ ID NO: 8
GWWRRAVAKVRK
(V7A)
SEQ ID NO: 9
GWWRRTAAKVRK
(HC(1-12)A8K12)
SEQ ID NO: 10
GWWRRTVAKVRK
(K9A)
SEQ ID NO: 11
GWWRRTVAAVRK
(V10A)
SEQ ID NO: 12
GWWRRTVAKARK
(R11A)
SEQ ID NO: 13
GWWRRTVAKVAK
(K12A)
SEQ ID NO: 14
GWWRRTVAKVRA
(HC(1-16))
SEQ ID NO: 15
GWWRRTVDKVRNAGRK
(HC(1-16)A8)
SEQ ID NO: 16
GWWRRTVAKVRNAGRK
(HC(2-16)A7)
SEQ ID NO: 17
WWRRTVAKVRNAGRK
(HC(1-12)A8)
SEQ ID NO: 18
GWWRRTVAKVRN
(HC(1-9)R8)
SEQ ID NO: 19
GWWRRTVRK
(HC(1-12)W6A8K12)
SEQ ID NO: 20
GWWRRWVAKVRK
(HC(1-12)K6A8K12)
SEQ ID NO: 21
GWWRRKVAKVRK
(HC(1-12)W6K8K12)
SEQ ID NO: 22
GWWRRWVKKVRK
(HC(1-12)K1W6K8K12)
SEQ ID NO: 23
KWWRRWVKKVRK
(HC(1-12)A3W6A8K12)
SEQ ID NO: 24
GWARRWVAKVRK
(HC(1-12)R1W6R8,9,12-NH₂)
SEQ ID NO: 25
RWWRRWVRRVRR

The invention will now be described by way of a non-limiting Examples with reference to the following figures in which:

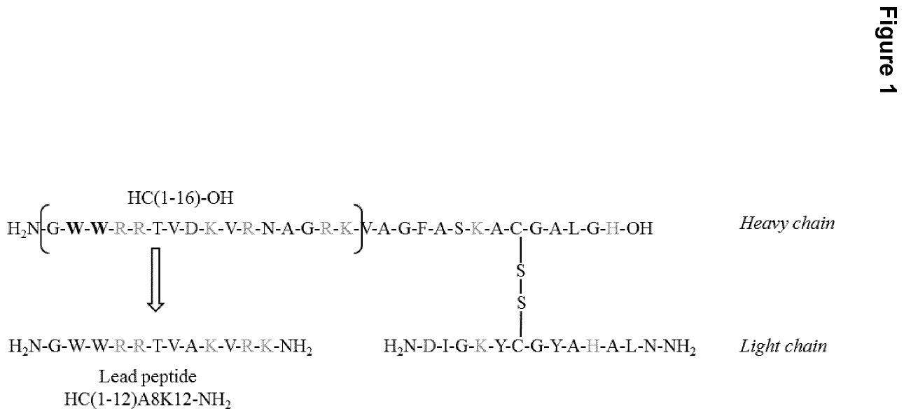

FIG. 1 depicts the amino acid sequence of EeCentrocin 1 and the lead peptide HC(1-12)A8K12.

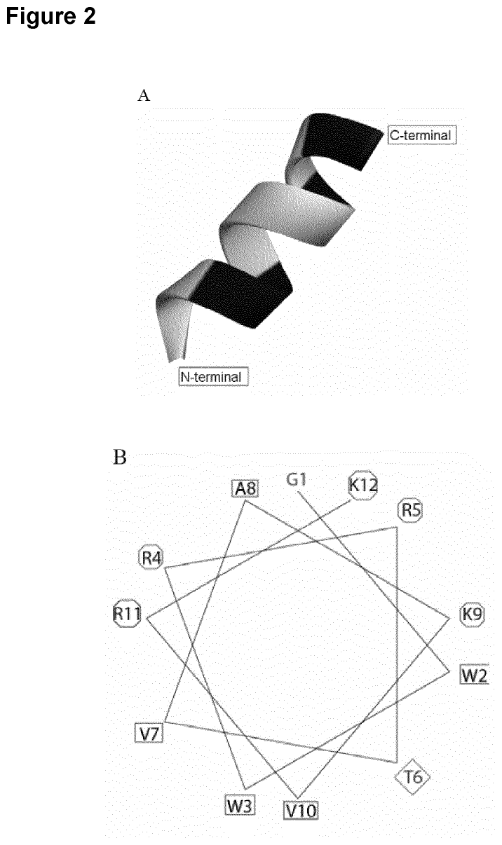

FIG. 2 shows the predicted secondary structure (A) and helical wheel projection (B) of the peptide HC(1-12)A8K12. The dark portions on FIG. 1A represent Arg/Lys residues. The octagons in the helical wheel projection (B) represent Arg/Lys residues, the square boxes represent hydrophobic residues and the diamond represents the Thr6 residue.

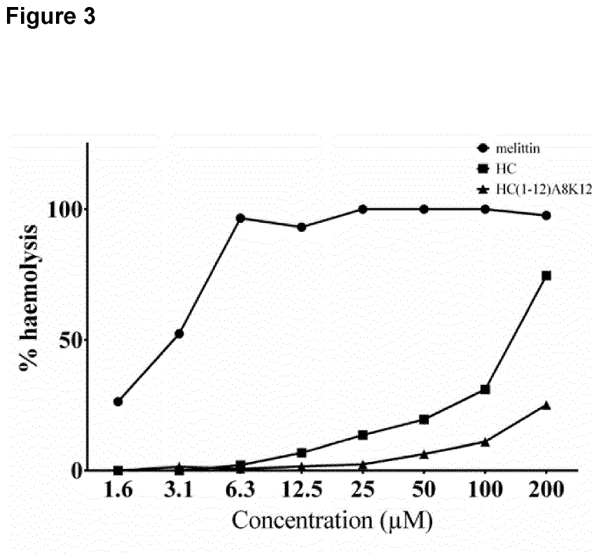

FIG. 3 shows haemolytic activity (% haemolysis) against human red blood cells of EeCentrocin 1 HC, the lead peptide HC(1-12)A8K12, and melittin.

### EXAMPLE 1

Increased microbial resistance to commercial antibiotics has led to an extensive search for novel antimicrobial agents to overcome this challenge. Antimicrobial peptides (AMPs) have the ability to kill bacterial pathogens and have therefore attracted interest as novel antimicrobial lead compounds. EeCentrocin 1 is a potent AMP, originally isolated from the marine sea urchin Echinus esculentus. The AMP has a hetero-dimeric structure with the pharmacophore located in its largest monomer (the heavy chain, HC), containing 30 amino acids. In the present study, the pharmacophore has been located within the HC and structure-activity relationship studies and sequence modification of the identified pharmacophore has been done. A lead peptide identified is superior in antifungal activity compared to the other peptides with minimal inhibitory concentrations (MICs) in the low micromolar range and also retains good antibacterial activity. In addition, the peptide displayed minor haemolytic activity.

Materials and Methods

Solid Phase Peptide Synthesis (SPPS)

The non-brominated heavy chain (HC) of EeCentrocin 1, the truncated peptide HC(1-16), and the modified peptide HC(1-16)A8 were synthesised commercially (GenicBio Ltd., Shanghai, China). The other peptides were synthesized by microwave assisted Fmoc-based solid phase peptide synthesis (Fmoc-SPPS). All Fmoc-amino acids and solvents were purchased from Sigma-Aldrich (MO, USA) whereas Rink amide ChemMatrix resin was obtained from Biotage (Uppsala, Sweden). The most efficient procedure involved using Rink amide ChemMatrix resin (loading 0.47-0.49 mmol/gram), which was swelled in N,N-dimethylformamide (DMF) in a 10 ml fritted reaction vial for 20 min with microwave heating at 70° C. Fmoc-amino acids (4.2 eq.) were dissolved in N-methyl-2-pyrrolidone (NMP) prior to in situ coupling with O-(6-chlorobenzotriazol-1-yl)-N,N,N′,N′-tetramethyluronium hexafluorophosphate (HCTU, 4.12 eq.) and N,N-diisopropylethylamine (DIEA, 8.4 eq.) as base, and coupling for five min with microwave heating at 75° C. Fmoc-Arg(Pbf)-OH was coupled at room temperature for 60 min to avoid δ-lactamisation of its side-chain and we found it necessary to double couple the N-terminal Gly-residue to avoid Gly-1 deletion peptides. Fmoc-cleavage was performed with a solution of 20% piperidine in DMF (4.5 ml for three min and repeated for 10 min) at room temperature, and the resin washed with DMF (4×4.5 ml for 0.45 min). After the final coupling and Fmoc-cleavage of the N-terminal Gly-residue, the resin was washed thoroughly with dichloromethane (DCM) and dried overnight in a desiccator. A 10 ml solution of 95% trifluoroacetic acid (TFA), 2.5% triisopropylsilane (TIS), and 2.5% H₂O was used as cleavage cocktail and added to the 10 ml fritted reaction vial with gentle stirring every hour for 3-3.5 h. The solution was filtered on a Supelco Visiprep vacuum manifold and the cleavage process was repeated with 5 ml of the cleavage cocktail for 0.5-1 h. The solution was concentrated in vacuo and ice-cold diethyl ether was added for precipitation of the crude peptide. The precipitated crude peptide was washed three times with ice-cold diethyl ether to remove traces of the cleavage cocktail.

High-Performance Liquid Chromatography

The peptides were purified by RP-HPLC using a Waters 2690 module equipped with a Waters 996 photodiode array detector and an XBridge C₁₈, 5 μm, 10×250 mm column (Waters, Mass. USA). The mobile phases consisted of buffer A: H₂O/0.1% TFA and buffer B1: 80% ACN/20% H₂O/0.1% TFA (Sigma-Aldrich). Depending on the individual peptide (hydrophobicity and co-eluting reagents), linear gradients for purification went from 5, 10, 15 or 17% buffer B to 35% buffer B in 24 min and with a flow of 2 ml/min for one min initially, followed by 5 ml/min during the run. The purity of all peptides was estimated to be above 95%.

Mass Spectrometry

Molecular weight and purity of the peptides were confirmed using a 6540B Q-TOF mass spectrometer with a dual ESI source, coupled to a 1290 Infinity UHPLC system, controlled by the MassHunter software (Agilent, Calif., USA). The peptides were separated using a Zorbax C₁₈, 2.1×50 mm, 1.8 μm column (Agilent). System details and typical parameters are found in Table C. A specific gradient running from 3-20% buffer B2 (ACN/0.1% formic acid) was applied for the determination of retention times of the 12-mer alanine-scan peptides.

TABLE C
Typical parameters for HPLC-MS HPLC system

| | |
|---|---|
| 1290 Infinity analytical binary | G4220A |
| pump with degasser | |
| 1290 Infinity TCC column oven | G1316C |
| 1290 Infinity Autosampler with | G4226A |
| thermostat | |
| Gradient | 5% to 60% buffer B2 |
| Flow-rate | 0.4 ml/min |
| Column temperature | 40° C. |

Q-TOF

| | |
|---|---|
| Dual ESI | |
| Positive ion mode ADC | 2 GHz |
| (analog-to-digital) acquisition | |
| Gas temperature | 300° C. |
| Drying gas | 8 L/min |
| Nebulizer gas | 35 Psig |
| Capillary voltage | 3.5 kV |
| Fragmentor | 175 V |
| Skimmer | 65 V |
| Drying and nebulizer gas | N₂ |
| Max range m/z for sample | 100-3200 |
| acquisition | |
| Reference mass | 121.050873, |
| | 922.009798 |

Software

| | |
|---|---|
| Masshunter Acquisition | Vesion B.06.01 Build |
| | 6.01.6157 |
| MassHunter Qualitative | Vesion B.07.00 Build |
| Analysis | 7.0.7024.29 service |
| | pack 1 |

Antibacterial Assay

The peptides were screened for antibacterial activity against two strains of Gram-positive and two strains of Gram-negative bacteria; Corynebacterium glutamicum (Cg, ATCC 13032), Staphylococcus aureus (Sa, ATCC 9144), Pseudomonas aeruginosa (Pa, ATCC 27853) and Escherichia coli (Ec, ATCC 25922).

Cultures stored at −80° C. in glycerol were transferred to Müller-Hinton plates (MH, Difco, Lawrence, Kans., USA) and incubated for 24 h at 35° C. A few colonies of each bacterial strain were transferred to 5 ml liquid MH medium and left shaking at room temperature overnight at 600 rpm. Cultures of actively growing bacteria (20 μl) were inoculated in 5 ml MH medium and left shaking for 2 h at room temperature. The antibacterial assays were performed as previously described by Sperstad, S. V., et al. ((2009) Mol. Immunol. 46, 2604-2612) with the following exception: bacterial cultures were diluted with medium to 2.5-3×10⁴ bacteria/ml concentrations. An aliquot of 50 μl (1250-1500 bacterial cells) was added to each well in 96-well Nunclon™ microtiter plates (Nagle Nunc Int., Denmark) preloaded with peptide solution (50 μl).

The microtiter plates were incubated for 24 h at 35° C. with optical density recorded every hour using an Envision 2103 multilabel reader, controlled by a Wallac Envision manager (PerkinElmer, Conn., USA). Minimum inhibitory concentration (MIC) was defined as a sample showing complete inhibition (as measured by optical density at 595 nm) compared to the negative (growth) controls, consisting of bacteria and water. Oxytetracycline (20 μM) served as a positive (inhibition) control.

The synthetic peptides were tested for antibacterial activity in concentrations ranging from 200 to 0.1 μM in two-fold dilutions. All tests were performed in triplicates.

Antifungal Assay

The synthetic peptides were also screened for antifungal activity against Candida albicans (ATCC 10231), Aureobasidium pullulans and Rhodotorula sp. (the last two were obtained from Professor Arne Tronsmo, The Norwegian University of Life Sciences, As, Norway). The antifungal assay was performed as previously described (Sperstad, S. V., et al. (2009) Dev. Comp. Immunol. 33, 583-591). Briefly, fungal spores were dissolved in potato dextrose broth (Difco, Lawrence, Kans., USA) to a concentration of 4×10⁵ spores/ml. The spores (50 μl) were inoculated on 96-well Nunclon™ microtiter plates containing the synthetic peptides (50 μl) dissolved in MQ-H₂O. Fungal growth and MIC (defined as the lowest concentration of peptide giving no visible growth) were determined visually after incubation for 24 h at room temperature. The negative (growth) control consisted of medium and fungal solution. The peptides were tested for activity in concentrations ranging from 100 to 0.1 μM in two-fold serial dilutions. All tests were performed in triplicates.

Haemolytic Assay

Selected synthesised peptide analogues were also screened for haemolytic activity using human red blood cells as described previously (Sperstad, S. V., et al. (2009) Dev. Comp. Immunol. 33, 583-591). The assay was performed on 96-well U-shaped microtiter plates (Nagle Nunc) with 50 μl peptide sample, 40 μl phosphate-buffered saline (PBS) and 10 μl red blood cells. After one hour of incubation at 37° C. in a shaker, the plate was centrifuged at 200 g for 5 min and the supernatants (60 μl) were carefully transferred to a new flat-bottomed polycarbonate microtiter plate (Nagle Nunc) and the release of haemoglobin (absorbance at 550 nm) was measured on a Synergy H1 multimode reader (BioTek, Vt., USA). Cell suspension added 0.05% Triton X-100 (Sigma-Aldrich, Mo., USA) in PBS served as positive (100% haemolysis) control and cell suspension added PBS served as negative (0% haemolysis, blank) control. The percent haemolysis was calculated using the formula [(Asample-Abaseline)/(Atriton-Abaseline)]×100. The cytotoxic peptide melittin (Sigma-Aldrich) was used as a positive control peptide and for comparison. The experiment was performed in triplicates with peptide concentrations ranging from 200 μM to 1.56 μM in two-fold dilutions.

Bioinformatics

Peptide properties were calculated with PEPCALC (http://pepcalc.com) from Innovagen, and helical wheel projections made with Pepwheel at the EMBOSS suite http://www.bioinformatics.nl/cgi-bin/emboss/pepwheel. Secondary structures were predicted using PEP-FOLD3 (Plamiable, A., et al. (2016) Nucleic Acids Res. 44, W449-W454) at the Mobyle portal and resulting figures were made with BIOVIA Discovery Studio Visualizer v4.5.0.15071. Homology search was performed with BLAST searching for non-redundant protein sequences at the National Centre for Biotechnological Information (NCBI) homepage (http://blast.ncbi.nlm.nih.gov/Blast.cgi) (Zhang, Z., et al. (2000) J. Comput. Biol. 7, 203-214).

Results and Discussion

Truncation of Non-Brominated HC

In silico modelling of the EeCentrocin 1 HC revealed that the N-terminal part of the sequence most likely forms an α-helix. In addition, the N-terminal part has an abundance of hydrophobic and positively charged residues. The last 14 C-terminal amino acids were removed from the HC of EeCentrocin 1, resulting in the peptide HC(1-16). The peptide contains two Trp and six positively charged (Arg/Lys) residues. Truncation led to reduced antibacterial activity, especially against S. aureus (the truncation experiments are presented in Table 1). However, the Gram-positive C. glutamicum was still sensitive to HC(1-16) albeit at slightly higher concentrations. An 8-fold decrease in activity was also observed against the Gram-negative test bacteria (Escherichia coli and Pseudomonas aeruginosa).

TABLE 1
Sequences and in vitro antibacterial (MIC, μM) activities of EeCentrocin 1 HC and truncated analogues.

| | | Amino acid sequence and position | | | | | | | | | | | | | | | | | | |
|---|---|---|---|---|---|---|---|---|---|---|---|---|---|---|---|---|---|---|---|---|
| Peptide | Mw (Da) | 1 | 2 | 3 | 4 | 5 | 6 | 7 | 8 | 9 | 10 | 11 | 12 | 13 | 14 | 15 | 16 | 17 | 18 | 19 |
| HC | 3255.7 | G | W | W | R | R | T | V | D | K | V | R | N | A | G | R | K | V | A | G |
| HC(1-16) | 1985.3 | G | W | W | R | R | T | V | D | K | V | R | N | A | G | R | K | | | |
| HC(1-16)A8 | 1940.3 | G | W | W | R | R | T | V | A | K | V | R | N | A | G | R | K | | | |
| HC(2-16)A7 | 1883.2 | | W | W | R | R | T | V | A | K | V | R | N | A | G | R | K | | | |
| HC(1-12)A8 | 1527.8 | G | W | W | R | R | T | V | A | K | V | R | N | | | | | | | |
| HC(1-12)A8K12 | 1541.9 | G | W | W | R | R | T | V | A | K | V | R | K | | | | | | | |
| HC(1-9)R8 | 1243.5 | G | W | W | R | R | T | V | R | K | | | | | | | | | | |

| | Amino acid sequence and position | | | | | | | | | | | | | MIC (μM) | | | |
|---|---|---|---|---|---|---|---|---|---|---|---|---|---|---|---|---|---|
| Peptide | 20 | 21 | 22 | 23 | 24 | 25 | 26 | 27 | 28 | 29 | 30 | C-term | Charge | Cg | Sa | Pa | Ec |
| HC | F | A | S | K | A | C | G | A | L | G | H | —OH | +6 | 0.4 | 3.1 | 1.6 | 0.8 |
| HC(1-16) | | | | | | | | | | | | —OH | +5 | 0.8 | 200 | 12.5 | 6.3 |
| HC(1-16)A8 | | | | | | | | | | | | —NH₂ | +7 | 0.8 | 12.5 | 3.1 | 3.1 |
| HC(2-16)A7 | | | | | | | | | | | | —NH₂ | +7 | 0.4 | 12.5 | 25 | 6.3 |
| HC(1-12)A8 | | | | | | | | | | | | —NH₂ | +5 | 1.6 | 50 | 6.3 | 6.3 |
| HC(1-12)A8K12 | | | | | | | | | | | | —NH₂ | +6 | 0.4 | 12.5 | 1.6 | 3.1 |
| HC(1-9)R8 | | | | | | | | | | | | —NH₂ | +5 | 3.1 | 100 | 25 | 50 |

MIC: Minimal Inhibitory Concentration

The negatively charged Asp8 residue was replaced by an Ala-residue and the C-terminal residue amidated in HC(1-16)A8. The peptide HC(1-16)A8 was antibacterial at low concentrations and clearly pointed to the importance of eliminating negatively charged groups. Thus, all subsequent synthesised AMPs were prepared with a C-terminal amide.

Early in the process, it was found that eliminating the N-terminal Gly1 was not beneficial for antibacterial activity as shown for the resulting peptide HC(2-16)A7. This peptide displayed a remarkable drop in antibacterial activity against the Gram-negative P. aeruginosa compared to the HC(1-16)A8 analogue. However, the activity towards the other test strains remained similar.

As the N-terminal truncation proved unsuccessful against P. aeruginosa, the focus was directed towards further truncation of the C-terminal end, producing the peptide HC(1-12)A8. The antibacterial activity of HC(1-12)A8 was somewhat reduced compared to the larger HC(1-16)A8 against all strains. In an attempt to improve the antibacterial activity of this 12-residue peptide, the C-terminal Arg-Lys-motif, which was recognised in the 16-residue peptides, was reinstated. This also made it possible to replace the original Asn12-residue, which could compromise peptide integrity by forming aspartimide side-products in SPPS involving chain-elongation through its side-chain and not the peptide-backbone. The resulting peptide HC(1-12)A8K12 (FIG. 1) was the most potent shortened AMP tested with antibacterial activities towards C. glutamicum and P. aeruginosa identical to the original HC peptide.

An analogue HC(1-9)R8 was synthesised to further shorten the peptide sequence and also reinstate the C-terminal Arg-Lys-motif. However, the potency of this 9-residue peptide was much lower than the previous peptides.

Accordingly, HC(1-12)A8K12 peptide was chosen as lead peptide, and represented an AMP two-fifths (40%) of the sequence-size (length) of the original HC (full-length HC).

Alanine-Scan of the Lead Peptide HC(1-12)A8K12

In order to investigate the importance of individual residues of the lead peptide, each amino acid was substituted by Ala and antibacterial activity was recorded for each peptide. The peptides were named (apart from the lead peptide, HC(1-12)A8K12) according to the original amino acid, position and substitution, i.e. the peptide where Gly was substituted with Ala in position 1, was named G1A (Table 2). Table 2 shows the antibacterial results from the Ala-scan on the lead peptide HC(1-12)A8K12.

TABLE 2
Antibacterial activity (MIC, μM) of alanine scan peptides derived from the lead peptide HC(1-12)A8K12. The nomenclature
indicates which amino acid has been exchanged with alanine (e.g. G1A: Gly1 is exchanged with Ala). The table also shows
sequences, molecular weights (Mw), retention time (RT, min) on a RP-HPLC column, and peptide net charge.

| | | Amino acid sequence and position | | | | | | | | | | | | | | | MIC (μM) | | | |
|---|---|---|---|---|---|---|---|---|---|---|---|---|---|---|---|---|---|---|---|---|
| Peptide | Mw (Da) | 1 | 2 | 3 | 4 | 5 | 6 | 7 | 8 | 9 | 10 | 11 | 12 | C-term | Charge | RT | Cg | Sa | Pa | Ec |
| G1A | 1555.9 | A | W | W | R | R | T | V | A | K | V | R | K | —NH₂ | +6 | 5.3 | 0.8 | 25 | 6.3 | 12.5 |
| W2A | 1426.7 | G | A | W | R | R | T | V | A | K | V | R | K | —NH₂ | +6 | 2.1 | 3.1 | 100 | 200 | 50 |
| W3A | 1426.7 | G | W | A | R | R | T | V | A | K | V | R | K | —NH₂ | +6 | 2.2 | 0.8 | 25 | 100 | 100 |
| R4A | 1456.7 | G | W | W | A | R | T | V | A | K | V | R | K | —NH₂ | +5 | 7.9 | 1.6 | 50 | 12.5 | 6.3 |
| R5A | 1456.7 | G | W | W | R | A | T | V | A | K | V | R | K | —NH₂ | +5 | 7.3 | 0.8 | 100 | 25 | 6.3 |
| T6A | 1511.8 | G | W | W | R | R | A | V | A | K | V | R | K | —NH₂ | +6 | 5.9 | 1.6 | 12.5 | 3.1 | 3.1 |
| V7A | 1513.8 | G | W | W | R | R | T | A | A | K | V | R | K | —NH₂ | +6 | 4.0 | 1.6 | 100 | 12.5 | 12.5 |
| HC(1-12)A8K12 | 1541.9 | G | W | W | R | R | T | V | A | K | V | R | K | —NH₂ | +6 | 5.2 | 0.4 | 12.5 | 1.6 | 3.1 |
| K9A | 1484.8 | G | W | W | R | R | T | V | A | A | V | R | K | —NH₂ | +5 | 7.1 | 1.6 | 100 | 6.3 | 6.3 |
| V10A | 1513.8 | G | W | W | R | R | T | V | A | K | A | R | K | —NH₂ | +6 | 4.1 | 1.6 | 100 | 6.3 | 12.5 |
| R11A | 1456.7 | G | W | W | R | R | T | V | A | K | V | A | K | —NH₂ | +5 | 6.8 | 1.6 | 100 | 12.5 | 12.5 |
| K12A | 1484.8 | G | W | W | R | R | T | V | A | K | V | R | A | —NH₂ | +5 | 6.9 | 0.8 | 50 | 6.3 | 6.3 |

MIC: Minimal Inhibitory Concentration.

All Ala-scan peptides displayed antibacterial activity, but to a different degree against the different strains. Overall, C. glutamicum was the most sensitive strain to the peptides. The other Gram-positive strain, S. aureus, was the least sensitive in the experiment, resisting all the AMPs in concentrations below 12.5 μM. Four AMPs were antibacterial towards S. aureus in concentrations <50 μM; the lead peptide (HC(1-12)A8K12), G1A, W3A and T6A, and an additional two AMPs were antibacterial towards S. aureus at 50 μM concentrations; R4A and K12A. Against the two Gram-negative strains (Escherichia coli and Pseudomonas aeruginosa) the lead peptide, T6A, K9A and K12A were antibacterial at concentrations ≤6.3 μM, whereas W2A and W3A were the least antibacterial with MICs ≥50 μM against the Gram-negative bacteria. The stand-out (i.e. best) performer in terms of antibacterial profile (antibacterial activity across the strains tested) was the lead peptide (HC(1-12)A8K12).

In general, all Ala-substitutions of the lead peptide resulted in reduced antibacterial activity. The peptide T6A was generally the most potent AMP after the lead peptide. However, while T6A was only marginally less potent than the lead peptide against P. aeruginosa, a four-fold dilution separated T6A and the lead peptide towards C. glutamicum. This observation indicated a selective drop in the antibacterial activity of T6A towards C. glutamicum. The lead peptide and T6A were different in that Thr is a polar residue without charge whereas Ala is smaller and more hydrophobic.

Replacement of the positively charged residues (R4A, R5A, K9A, R11A and K12A) with Alanine resulted in 2-8 fold reduction of antibacterial activity against all strains tested. Of notice, Lys12, which is positioned at the C-terminal end of the peptide, seemed to be the least important positively charged residue.

An interesting pair when considering individual amino acid substitutions were the Tryptophan substitutions represented by the peptides W2A and W3A. In the current Ala-scan, Ala-substitutions of W2 or W3 resulted in a dramatic loss of antibacterial activity. W3A and G1A have similar potency towards the Gram-positive bacteria, but W3A is noticeably less potent than G1A towards the Gram-negative bacteria. The one AMP that was consistently the least potent towards all strains was W2A, which indicates that Trp2 may be a more important residue for antibacterial activity than Trp3. The exception was the data against E. coli where W2A was somewhat more potent than W3A. As shown in Table 2, the retention times (hydrophobicity) for W2A and W3A on a C18 RP-HPLC column was highly reduced compared to the lead peptide HC(1-12)A8K12. This illustrated the importance of the hydrophobic character contributed by the two Tryptophan-residues (both located in the hydrophobic face of the α-helix) to the antibacterial activity.

Replacement of the polar uncharged Gly1 with the small hydrophobic Ala (G1A) resulted in 2-4 fold increase in MIC against all bacterial strains compared to the lead peptide. In the helical wheel projection (FIG. 2B) Gly1 is positioned in the polar and cationic face of the α-helix. In addition, glycine does not have a side chain and may therefore provide increased flexibility to this region of the peptide.

Ala-substitution of the hydrophobic Val7 (V7A) or Val10 (V10A), both positioned in the hydrophobic region of the α-helix (FIG. 2B), resulted in 4-8 fold decrease in activity against all bacterial strains tested. Valine contains an isopropyl side chain, in contrast to the methyl side chain displayed by Alanine. Replacement of Val with Ala would therefore slightly decrease the size of the hydrophobic sector and thereby the amphipacity of the peptide. As shown in Table 2, the retention times are reduced for V7A and V10A, indicating reduced hydrophobicity for these two peptides compared to the lead peptide.

In a further experiment, the peptide GWWRRWVAKVRK (amidated at C-terminus) was also tested for antibacterial activity. This peptide differs from the lead peptide HC(1-12)A8K12 in that the T at position 6 has been replaced by a W. This peptide showed good antibacterial activity, with a MIC against C. glutamicum of 0.8 μM, a MIC against S. aureus of 3.1 μM, a MIC against P. aeruginosa of 1.6 μM and a MIC against Escherichia coli of 1.6 μM.

Antifungal Activity

The synthesised peptides were subjected to antifungal screening against the moulds A. pullulans and Rhodotorula sp., and the yeast C. albicans. Surprisingly, the lead peptide HC(1-12)A8K12 was superior in activity compared to the other peptides, including the full HC peptide (Table 3). The lead peptide was antifungal at concentrations (MICs) ranging from 1.6-6.3 μM, whereas the other peptides had MICs from 12.5 μM and upwards. Thus, the lead peptide HC(1-12)A8K12 was the stand-out (i.e. best) performer in terms of antifungal activity. In addition, whereas the full HC is more potent against bacteria than fungi, the lead peptide HC(1-12)A8K12 is equally active against both types of microorganisms, i.e. showing broad-spectrum antimicrobial activity.

In a further experiment, the peptide GWWRRWVAKVRK (amidated at C-terminus) was also tested for antifungal activity. This peptide differs from the lead peptide HC(1-12)A8K12 in that the T at position 6 has been replaced by a W. This peptide showed good antifungal activity, with a MIC against C. albicans of 12.5 μM and a MIC against Rhodotorula sp. of 1.6 μM.

Haemolytic Activity

To test whether the lead peptide or the other peptides were cytotoxic, their haemolytic activity on human red blood cells was determined. The data obtained indicated a correlation between the antibacterial and the haemolytic activities. The peptides showing highest haemolytic activities were the lead peptide and the full HC, with 25% and 75% haemolysis at 200 μM respectively (Table 3 and FIG. 3). At concentrations closer to the MIC-values, the haemolytic activity of the lead peptide is negligible. All the other peptides displayed minor (5%) or no haemolytic activity at 200 μM. By contrast, the bee venom peptide melittin displayed 100% haemolysis at concentrations as low as 6.3 μM (FIG. 3).

TABLE 3
Antifungal (MIC, μM) and haemolytic activities (% haemolysis at
200 μM and 25 μM) of EeCentrocin 1 HC, truncated analogues
and alanine scan peptides.

| | MIC (μM) | | | | |
|---|---|---|---|---|---|
| | C. | A. | Rhodotorula | Haemolysis (%) | |
| Peptide | albicans | pullulans | sp. | 200 μM | 25 μM |
| HC | 100 | 12.5 | 12.5 | 74.6 | 13.5 |
| HC(1-16) | 100 | 50 | 25 | 0.0 | 0.0 |
| HC(1-16)A8 | Nt | Nt | Nt | 5.0 | 0.0 |
| G1A | 25 | 50 | 12.5 | 1.6 | 0.0 |
| W2A | 50 | 50 | 25 | 0.0 | 0.0 |
| W3A | 50 | 50 | 25 | 0.0 | 0.0 |
| R4A | 25 | 50 | 50 | 3.6 | 0.0 |
| R5A | 25 | 25 | 12.5 | 0.0 | 0.0 |
| T6A | 25 | 25 | 12.5 | 4.1 | 0.9 |
| V7A | 25 | 25 | 12.5 | 0.0 | 0.0 |
| HC(1-12)A8K12 | 3.1 | 6.3 | 1.6 | 25.1 | 2.4 |
| K9A | 25 | 25 | 12.5 | 5.1 | 0.0 |
| V10A | 25 | 50 | 12.5 | 1.0 | 0.0 |
| R11A | 25 | 50 | 12.5 | 0.0 | 0.0 |
| K12A | 25 | 50 | 12.5 | 0.0 | 0.0 |

MIC: Minimal Inhibitory Concentration
Nt: Not tested

### CONCLUSION

Natural AMPs can be challenging to synthesise due to both large size and post-translational modifications. However, those properties are not always necessary for the antimicrobial activity as the pharmacophore may be located in only a minor sequence-motif and post-translational modifications can have a variety of purposes. In the present study, the pharmacophore of the antimicrobial peptide EeCentrocin 1 HC was located to the N-terminal part of the sequence. Truncation of EeCentrocin 1 HC, and selected amino acid substitutions, combined with C-terminal amidation, led to the production of a 12-residue lead peptide with potent antibacterial and antifungal activities. The lead peptide HC(1-12)A8K12 possibly forms an α-helical structure. This is supported by helical wheel projection and secondary structure predictions. The peptide also displays low haemolytic activity at the MIC, making it a promising lead peptide for further drug development.

### EXAMPLE 2

Further peptides were synthesised and tested for their antimicrobial activity. The methodology used is as per the description in Example 1.

These peptides are named HC(1-12)W6A8K12, HC(1-12)A3W6A8K12, HC(1-12)K6A8K12, HC(1-12)W6K8K12, HC(1-12)K1W6K8K12 and HC(1-12)R1W6R8,9,12-NH2 and their amino acid sequences are set forth in Table 4 below.

The antifungal and antibacterial MIC data (μM) for these peptides is presented in Table 4 below. A new batch (new synthesis) of the peptide HC(1-12)A8K12 was also prepared and tested.

Data from Example 1 is also included in Table 4 below.

TABLE 4

| | # | | MIC (μM) | | | | | | |
|---|---|---|---|---|---|---|---|---|---|
| Peptide Name | aa | Sequence | Cg | Sa | Pa | Ec | Ca | Ap | Rh |
| G1A | 12 | AWWRRTVAKVRK-NH₂ | 0.8 | 25 | 6.3 | 12.5 | 25 | 50 | 12.5 |
| W2A | 12 | GAWRRTVAKVRK-NH₂ | 3.1 | 100 | 200 | 50 | 50 | 50 | 25 |
| W3A | 12 | GWARRTVAKVRK-NH₂ | 0.8 | 25 | 100 | 100 | 50 | 50 | 25 |
| R4A | 12 | GWWARTVAKVRK-NH₂ | 1.6 | 50 | 12.5 | 6.3 | 25 | 50 | 50 |
| R5A | 12 | GWWRATVAKVRK-NH₂ | 0.8 | 100 | 25 | 6.3 | 25 | 25 | 12.5 |
| T6A | 12 | GWWRRAVAKVRK-NH₂ | 1.6 | 12.5 | 3.1 | 3.1 | 25 | 25 | 12.5 |
| V7A | 12 | GWWRRTAAKVRK-NH₂ | 1.6 | 100 | 12.5 | 12.5 | 25 | 25 | 12.5 |
| HC(1-12)A8K12 | 12 | GWWRRTVAKVRK-NH₂ | 0.4 | 12.5 | 1.6 | 3.1 | 3.1 | 6.3 | 1.6 |
| K9A | 12 | GWWRRTVAAVRK-NH₂ | 1.6 | 100 | 6.3 | 6.3 | 25 | 25 | 12.5 |
| V10A | 12 | GWWRRTVAKARK-NH₂ | 1.6 | 100 | 6.3 | 12.5 | 25 | 50 | 12.5 |
| R11A | 12 | GWWRRTVAKVAK-NH₂ | 1.6 | 100 | 12.5 | 12.5 | 25 | 50 | 12.5 |
| K12A | 12 | GWWRRTVAKVRA-NH₂ | 0.8 | 50 | 6.3 | 6.3 | 25 | 50 | 12.5 |
| HC(1-12)A8K12 + 0.9% NaCl | 12 | GWWRRTVAKVRK-NH₂ | 0.4 | 6.3 | 6.3 | 6.3 | | | |
| | | New peptides | | | | | | | |
| HC(1-12)A8K12 | 12 | GWWRRTVAKVRK-NH₂ | 0.8 | 25 | 3.1 | 6.3 | 12.5 | 25 | 3.1 |
| (new synthesis) | | | | | | | | | |
| HC(1-12)W6A8K12 | 12 | GWWRRWVAKVRK-NH₂ | 0.8 | 3.1 | 1.6 | 1.6 | 12.5 | 50 | 3.1 |
| HC(1-12)A3W6A8K12 | 12 | GWARRWVAKVRK-NH₂ | 0.8 | 50 | 12.5 | 6.3 | 6.3 | 25 | 6.3 |
| HC(1-12)K6A8K12 | 12 | GWWRRKVAKVRK-NH₂ | 1.6 | 12.5 | 50 | 12.5 | 50 | 25 | 25 |
| HC(1-12)W6K8K12 | 12 | GWWRRWVKKVRK-NH₂ | 1.6 | 3.1 | 3.1 | 6.3 | 25 | 12.5 | 6.3 |
| HC(1-12)K1W6K8K12 | 12 | KWWRRWVKKVRK-NH₂ | 0.4 | 6.3 | 1.6 | 3.1 | 6.3 | 25 | 3.1 |
| HC(1-12)R1W6R8,9,12-NH2 | 12 | RWWRRWVRRVRR-NH₂ | 0.8 | 3.1 | 3.1 | 3.1 | 12.5 | 25 | 3.1 |

The data under the heading “New peptides” in Table 4 demonstrate that peptides HC(1-12)A8K12, HC(1-12)W6A8K12, HC(1-12)W6K8K12, HC(1-12)K1W6K8K12 and HC(1-12)R1W6R8,9,12-NH2 show good antimicrobial activity. Peptides HC(1-12)W6A8K12, HC(1-12)W6K8K12, HC(1-12)K1W6K8K12 and HC(1-12)R1W6R8,9,12-NH2 show particularly good activity against S. aureus (Sa) relative to other peptides.

The peptide HC(1-12)A8K12 (initial batch as reported in Example 1) was also tested for antibacterial activity in growth medium with 0.9% NaCI (which corresponds to 154 mM NaCl, physiological saline). The results show that activity against Gram positive bacteria (Cg and Sa) was retained and the activity against Gram-negative bacteria (Pa and Ec) was only slightly reduced. Many antimicrobial peptides lose their activity when exposed to salt-rich environments, like mucus (a problem for cystic fibrosis patients). Thus, the good activity observed in this experiment in the presence of 0.9% NaCI is beneficial.

1. A peptide that is 12-16 amino acids in length, wherein said peptide comprises an amino acid (AA) sequence of formula (IA) SEQ ID NO:27
(IA)
(SEQ ID NO: 27)
AA₁-AA₂-AA₃-AA₄-AA₅-AA₆-AA₇-AA₈-AA₉-AA₁₀-AA₁₁-AA₁₂
wherein
AA₁ is an amino acid that has a hydrophobicity that is less than or equal to the hydrophobicity of glycine and is not an anionic amino acid;
AA₂ and AA₃ are each an amino acid with a hydrophobic R group, said R group having at least 4 non-hydrogen atoms;
AA₄, AA₅, AA₉, AA₁₁ and AA₁₂ are each a cationic amino acid;
AA₆is an uncharged amino acid;
AA₈ and AA₁₀ are each an amino acid that is not an anionic amino acid; and
AA₇ is an amino acid with a hydrophobic R group, said R group having at least 3 non-hydrogen atoms;
or a peptidomimetic thereof.

2. A peptide or peptidomimetic of claim 1, wherein AA₁ is a cationic amino acid or an uncharged amino acid, preferably an uncharged amino acid.

3. A peptide or peptidomimetic of claim 1 or claim 2, wherein AA₁ is selected from the group consisting of G, T, S, N, Q, H, K and R.

4. A peptide or peptidomimetic of any one of claims 1 to 3, wherein AA₁ is an uncharged amino acid selected from the group consisting of G, T, S, N and Q.

5. A peptide or peptidomimetic of any one of claims 1 to 4, wherein AA₁ is G.

6. A peptide or peptidomimetic of any one of claims 1 to 3, wherein AA₁ is a cationic amino, preferably lysine (K) or arginine (R), but optionally histidine (H) or any non-genetically coded or modified amino acid carrying a positive charge at pH 7.0.

7. A peptide or peptidomimetic of any one of claim 1 to 3 or 6, wherein AA₁ is a cationic amino acid selected from the group consisting of H, K and R, preferably K and R, more preferably K.

8. A peptide or peptidomimetic of any one of claims 1 to 7, wherein at least one of AA₂ and AA₃ has at least 7, preferably at least 9, non-hydrogen atoms in its R group, more preferably both AA₂ and AA₃ have R groups having at least 7, preferably at least 9, non-hydrogen atoms.

9. A peptide or peptidomimetic of any one of claims 1 to 7, wherein one or both, preferably both, AA₂ and AA₃ has a hydrophobic R group that has a mass of >90Da.

10. A peptide or peptidomimetic of any one of claims 1 to 9, wherein the hydrophobic R group of AA₂ and/or AA₃ contains a hetero atom such as O, N or S, preferably there is no more than one heteroatom, preferably it is nitrogen.

11. A peptide or peptidomimetic of any one of claims 1 to 10, wherein AA₂ and AA₃ are each independently selected from the group consisting of W, F, Y, L and I, preferably, AA₂ and AA₃ are each independently selected from the group consisting of W, F and Y.

12. A peptide or peptidomimetic of any one of claims 1 to 11, wherein at least one, preferably both, of AA₂ and AA₃ is W, F or Y.

13. A peptide or peptidomimetic of any one of claims 1 to 12, wherein AA₂ and/or AA₃ is W, preferably both AA₂ and AA₃ is W.

14. A peptide or peptidomimetic of any one of claims 1 to 13, wherein AA₄, AA₅, AA₉, AA₁₁ and AA₁₂ are each a cationic amino acid independently selected from the group consisting of K, R, H and any non-genetically coded or modified amino acid carrying a positive charge at pH 7.0.

15. A peptide or peptidomimetic of any one of claims 1 to 14, wherein AA₄, AA₅, AA₉, AA₁₁ and AA₁₂ are each independently selected from the group consisting of K, R and H, preferably R and K.

16. A peptide or peptidomimetic of any one of claims 1 to 15, wherein AA₄ is R and/or AA₅ is R and/or AA₉ is K and/or AA₁₁ is R and/or AA₁₂ is K.

17. A peptide or peptidomimetic of any one of claims 1 to 16, wherein AA₄ is R, AA₅ is R, AA₉ is K, AA₁₁ is R and AA₁₂ is K.

18. A peptide or peptidomimetic of any one of claims 1 to 17, wherein AA₇ has a hydrophobic R group that has at least 4 non-hydrogen atoms, or at least 7 non-hydrogen atoms, or at least 9 non-hydrogen atoms.

19. A peptide or peptidomimetic of any one of claims 1 to 18, wherein AA₇ is an amino acid in accordance with the definitions of AA₂ or AA₃ in any preceding claim.

20. A peptide or peptidomimetic of any one of claims 1 to 19, wherein AA₇ is selected from the group consisting of W, F, Y, L, I, V, P and M, preferably selected from the group consisting of W, F, Y, L, I and V.

21. A peptide or peptidomimetic of any one of claim 1 to 17 or 20, wherein AA₇ is V.

22. A peptide or peptidomimetic of any one of claims 1 to 21, wherein one or both of AA₈ and AA₁₀ is an amino acid in accordance with the definitions of AA₁, AA₂, AA₃, AA₄, AA₅, AA₇, AA₉, AA₁₁ or AA₁₂ in any preceding claim.

23. A peptide or peptidomimetic of any one of claims 1 to 22, wherein one or both of AA₈ and AA₁₀ is an uncharged amino acid, preferably selected from the group consisting of W, F, Y, L, I, V, P, M, C, A, G, T, S, N and Q.

24. A peptide or peptidomimetic of any one of claims 1 to 23, wherein AA₆ is selected from the group consisting of W, F, Y, L, I, V, P, M, C, A, G, T, S, N and Q.

25. A peptide or peptidomimetic of any one of claims 1 to 22, wherein one or both of AA₈ and AA₁₀ is a cationic amino acid, preferably H, K or R, more preferably K or R.

26. A peptide or peptidomimetic of any one of claims 1 to 25, wherein AA₈ and AA₁₀ are each independently selected from the group consisting of W, F, Y, L, I, V, P, M, C, A, G, T, S, N, Q, H, K and R, preferably each independently selected from the group consisting of W, F, Y, L, I, V, M, A, G, T, S, N, Q, H, K and R.

27. A peptide or peptidomimetic of any one of claims 1 to 26, wherein AA₆ is selected from the group consisting of W, F, Y, L, I, V, M, A, G, T, S, N and Q.

28. A peptide or peptidomimetic of any one of claims 1 to 27, wherein AA₆ is an amino acid in accordance with a definition of AA₁ in claim 4 or claim 5, preferably AA₆ is selected from the group consisting of T, S, N and Q.

29. A peptide or peptidomimetic of any one of claims 1 to 27, wherein AA₆ is an amino acid that has a hydrophobicity that is greater than or equal to the hydrophobicity of glycine, preferably W or A, more preferably W.

30. A peptide or peptidomimetic of any one of claim 1 to 27 or 29, wherein AA₆ is an amino acid in accordance with the definition of AA₂, AA₃ or AA₇ in any preceding claim.

31. A peptide or peptidomimetic of any one of claim 1 to 27, 29 or 30, wherein AA₆ is selected from the group consisting of W, F, Y, L, I, V, P or M, or is selected from the group consisting of W, F, Y, L or I, or is selected from the group consisting of W, F or Y, or is W.

32. A peptide or peptidomimetic of any one of claims 1 to 27, wherein AA₆ is selected from the group consisting of T, S, N, Q, W or A, preferably selected from the group consisting of T, S, N, Q and W or T, S, N or Q, more preferably T or S.

33. A peptide or peptidomimetic of any one of claim 1 to 27 or 32, wherein AA₆ is selected from the group consisting of T, A or W.

34. A peptide or peptidomimetic of any one of claim 1 to 27, 32 or 33, wherein AA₆ is selected from the group consisting of T or W.

35. A peptide or peptidomimetic of any one of claims 1 to 27 or 32 to 34, wherein AA₆ is T.

36. A peptide or peptidomimetic of any one of claims 1 to 27 or 32 to 34, wherein AA₆ is W.

37. A peptide or peptidomimetic of any one of claim 1 to 27 or 32 or 33, wherein AA₆ is A.

38. A peptide or peptidomimetic of any one of claims 1 to 37, wherein AA₈ is an amino acid in accordance with a definition of AA₁, AA₂, AA₃, AA₄, AA₅, AA₇, AA₉, AA₁₁ or AA₁₂ in any preceding claim.

39. A peptide or peptidomimetic of any one of claims 1 to 38, wherein AA₈ is an amino acid in accordance with a definition of AA₁ in any preceding claim, preferably AA₈is selected from the group consisting of T, S, N, Q, H, K and R.

40. A peptide or peptidomimetic of any one of claims 1 to 38, wherein AA₈ is an amino acid that has a hydrophobicity that is greater than or equal to the hydrophobicity of glycine.

41. A peptide or peptidomimetic of any one of claims 1 to 39, wherein AA₈ is a cationic amino acid in accordance with the definition of AA₄, AA₅, AA₉, AA₁₁ and AA₁₂in any preceding claim.

42. A peptide or peptidomimetic of any one of claim 1 to 39 or 41, wherein AA₈ is selected from the group consisting of H, K and R, preferably K and R, more preferably AA₈ is K.

43. A peptide or peptidomimetic of any one of claims 1 to 39, wherein AA₈ is uncharged.

44. A peptide or peptidomimetic of any one of claim 1 to 39 or 43, wherein AA₈ is selected from the group consisting of T, S, N, Q or A.

45. A peptide or peptidomimetic of any one of claim 1 to 39, 43 or 44, wherein AA₈ is A.

46. A peptide or peptidomimetic of any one of claims 1 to 45, wherein AA₁₀ is an amino acid in accordance with a definition of AA₁, AA₂, AA₃, AA₄, AA₅, AA₇, AA₉, AA₁₁ or AA₁₂ in any preceding claim.

47. A peptide or peptidomimetic of any one of claims 1 to 46, wherein AA₁₀ is an amino acid in accordance with a definition of AA₁ in any preceding claim, preferably AA₁₀ is selected from the group consisting of T, S, N, Q, H, K and R.

48. A peptide or peptidomimetic of any one of claims 1 to 46, wherein AA₁₀ is uncharged.

49. A peptide or peptidomimetic of any one of claims 1 to 46, wherein AA₁₀ is an amino acid that has a hydrophobicity that is greater than or equal to the hydrophobicity of glycine.

50. A peptide or peptidomimetic of any one of claim 1 to 46, 48 or 49, wherein AA₁₀ is not A.

51. A peptide or peptidomimetic of any one of claims 1 to 46, wherein AA₁₀ is an amino acid in accordance with a definition of AA₇ any preceding claim.

52. A peptide or peptidomimetic of any one of claims 1 to 46, wherein AA₁₀ is an amino acid in accordance with a definition of AA₂ or AA₃ in any preceding claim.

53. A peptide or peptidomimetic of any one of claim 1 to 46, 51 or 52, wherein AA₁₀ is selected from the group consisting of W, F, Y, L, I, V, P and M, preferably selected from the group consisting of W, F, Y, L, I and V.

54. A peptide or peptidomimetic of any one of claim 1 to 46 or 51 or 53, wherein AA₁₀ is V.

55. A peptide or peptidomimetic of any one of claims 1 to 46, wherein AA₁₀ is a cationic amino acid in accordance with the definition of AA₄, AA₅, AA₉, AA₁₁ and AA₁₂in any preceding claim.

56. A peptide or peptidomimetic of any one of claim 1 to 46 or 55, wherein AA₁₀ is selected from the group consisting of H, K and R, preferably selected from the group consisting of K and R.

57. A peptide or peptidomimetic of any preceding claim, wherein AA₆ is T, A or W, preferably T or W, and/or AA₈ is A or K or R, preferably A, and/or AA₁₀ is V.

58. A peptide or peptidomimetic of any preceding claim, wherein AA₆ is T, AA₈ is A and AA₁₀ is V.

59. A peptide of claim 1, wherein said peptide comprises, or consists of, the amino acid sequence GWWRRTVAKVRK (SEQ ID NO:10), or a peptidomimetic thereof.

60. A peptide of claim 1, wherein said peptide is 12 amino acids in length and consists of the amino acid sequence GWWRRTVAKVRK (SEQ ID NO:10).

61. A peptide of claim 1, wherein said peptide comprises, or consists of, the amino acid sequence GWWRRWVAKVRK (SEQ ID NO:20), or a peptidomimetic thereof.

62. A peptide of claim 1, wherein said peptide is 12 amino acids in length and consists of the amino acid sequence GWWRRWVAKVRK (SEQ ID NO:20).

63. A peptide of claim 1, wherein said peptide comprises, or consists of, the amino acid sequence GWWRRAVAKVRK (SEQ ID NO:8), or a peptidomimetic thereof.

64. A peptide of claim 1, wherein said peptide is 12 amino acids in length and consists of the amino acid sequence GWWRRAVAKVRK (SEQ ID NO:8).

65. A peptide of claim 1, wherein said peptide comprises, or consists of, the amino acid sequence GWWRRWVKKVRK (SEQ ID NO:22), or a peptidomimetic thereof.

66. A peptide of claim 1, wherein said peptide is 12 amino acids in length and consists of the amino acid sequence GWWRRWVKKVRK (SEQ ID NO:22).

67. A peptide of claim 1, wherein said peptide comprises, or consists of, the amino acid sequence KWWRRWVKKVRK (SEQ ID NO:23), or a peptidomimetic thereof.

68. A peptide of claim 1, wherein said peptide is 12 amino acids in length and consists of the amino acid sequence KWWRRWVKKVRK (SEQ ID NO:23).

69. A peptide of claim 1, wherein said peptide comprises, or consists of, the amino acid sequence RWWRRWVRRVRR (SEQ ID NO:25), or a peptidomimetic thereof.

70. A peptide of claim 1, wherein said peptide is 12 amino acids in length and consists of the amino acid sequence RWWRRWVRRVRR (SEQ ID NO:25).

71. A peptide or peptidomimetic of any preceding claim, wherein said peptide comprises, or consists of, the amino acid sequence GWWRRTVAKVRK (SEQ ID NO:10), or a sequence substantially homologous thereto, wherein said substantially homologous sequence contains 1, 2 or 3 amino acid substitutions compared to the given amino acid sequence (SEQ ID NO:10), and wherein
(i) if the G at position 1 is replaced, the replacement amino acid is in accordance with AA₁ as defined in any preceding claim;
(ii) if one or both of the W residues at positions 2 and 3 is replaced, the replacement amino acid is in accordance with AA₂ or AA₃ as defined in any preceding claim;
(iii) if one or more of the residues at positions 4, 5, 9, 11 and 12 is replaced, the replacement amino acid is in accordance with AA₄, AA₅, AA₉, AA₁₁ or AA₁₂ as defined in any preceding claim;
(iv) if one or more of the residues at positions 6, 8 or 10 is replaced, the replacement amino acid is in accordance with AA₆, AA₈or AA₁₀, as defined in any preceding claim; and
(v) if the V at position 7 is replaced, the replacement amino acid is in accordance with AA₇ as defined in any preceding claim.

72. A peptide or peptidomimetic of any preceding claim, wherein said peptide or peptidomimetic is 12 amino acids in length.

73. A peptide or peptidomimetic of any preceding claim, wherein the molecule is a peptide.

74. A peptide or peptidomimetic of any preceding claim, wherein said peptide or peptidomimetic is amidated at the C-terminus.

75. A formulation comprising a peptide or peptidomimetic as defined in any one of the preceding claims in admixture with a suitable diluent, carrier or excipient.

76. The formulation of claim 75, which is a pharmaceutical formulation.

77. A peptide or peptidomimetic of any one of claims 1 to 74 for use in therapy.

78. A peptide or peptidomimetic of any one of claims 1 to 74 for use in the treatment of a microbial infection, preferably a bacterial or fungal infection.

79. Use of a peptide or peptidomimetic as defined in any one of claims 1 to 74 as an antimicrobial agent, preferably an antibacterial or antifungal agent.

80. A peptide or peptidomimetic of any one of claims 1 to 74 for use in the treatment of cancer.

81. A method of treating a microbial infection, preferably a bacterial or fungal infection, said method comprising administering to a patient in need thereof a therapeutically effective amount of a peptide or peptidomimetic as defined in any one of claims 1 to 74.

82. A method of treating cancer, said method comprising administering to a patient in need thereof a therapeutically effective amount of a peptide or peptidomimetic as defined in any one of claims 1 to 74.

83. Use of a peptide or peptidomimetic as defined in any one of claims 1 to 74 in the manufacture of a medicament for treating a microbial infection, preferably a bacterial or fungal infection.

84. Use of a peptide or peptidomimetic as defined in any one of claims 1 to 74 in the manufacture of a medicament for treating cancer.
